(19) United States
(12) Patent Application Publication
(10) Pub. No.: US 2010/0130515 A1
(43) Pub. Date: May 27, 2010

(54) Polymorphs and solvates of a pharmaceutical and method of making

(75) Inventors: Slawomir Janicki, North Chelmsford, MA (US); Hexi Chang, Belmont, MA (US); Weirong Chen, Waltham, MA (US); William F. Kiesman, Wayland, MA (US); Benjamin Lane, Chelsea, MA (US); Richard Todd, Winnersh (GB)

Correspondence Address: STEPTOE & JOHNSON LLP, 1330 CONNECTICUT AVE., NW, WASHINGTON, DC, 20036, US

(73) Assignees: BIOGEN IDEC INC., Cambridge, MA (US); VERNALIS RESEARCH LIMITED, Winnersh (GB)

(21) Appl. No.: 12/458,436

(22) Filed: Jul. 13, 2009

Related U.S. Application Data
(63) Continuation of application No. PCT/US2008/050268, filed on Jan. 4, 2008.
(60) Provisional application No. 60/883,588, filed on Jan. 5, 2007.

(51) Int. Cl. A61K 31/519; C07D 487/04; A61P 25/16; A61P 25/24; A61P 25/00

(52) U.S. Cl. 514/261.1; 544/254

(57) ABSTRACT

Polymorphic and solvated forms of solid 3-(4-amino-3-methylbenzyl)-7-(furan-2-yl)-3H-[1,2,3]triazolo[4,5-d]pyrimidin-5-amine, and methods of making them, are described.

### CLAIM OF PRIORITY

This application claims priority under 35 USC 120 to International Application No. PCT/US2008/050268, filed on Jan. 4, 2008, which claims priority to provisional U.S. Patent Application No. 60/883,588, filed Jan. 5, 2007, each of which is incorporated by reference in its entirety.

### TECHNICAL FIELD

The present invention relates to polymorphs and solvates of a pharmaceutical, and methods of making them.

### BACKGROUND

Movement disorders constitute a serious health problem, especially among the elderly. These movement disorders can often be the result of brain lesions. Disorders involving the basal ganglia which result in movement disorders include Parkinson's disease, Huntington's chorea and Wilson's disease. Furthermore, dyskinesias often arise as sequelae of cerebral ischaemia and other neurological disorders.

There are four classic symptoms of Parkinson's disease: tremor, rigidity, akinesia and postural changes. The disease is also commonly associated with depression, dementia and overall cognitive decline. Parkinson's disease has a prevalence of 1 per 1,000 of the total population. The incidence increases to 1 per 100 for those aged over 60 years. Degeneration of dopaminergic neurones in the substantia nigra and the subsequent reductions in interstitial concentrations of dopamine in the striatum are critical to the development of Parkinson's disease. Some 80% of cells from the substantia nigra can be destroyed before the clinical symptoms of Parkinson's disease become apparent.

Some strategies for the treatment of Parkinson's disease are based on transmitter replacement therapy (L-dihydroxyphenylacetic acid (L-DOPA)), inhibition of monoamine oxidase (e.g., Deprenyl™), dopamine receptor agonists (e.g., bromocriptine and apomorphine) and anticholinergics (e.g., benztrophine, orphenadrine). Transmitter replacement therapy may not provide consistent clinical benefit, especially after prolonged treatment when “on-off” symptoms develop. Furthermore, such treatments have also been associated with involuntary movements of athetosis and chorea, nausea and vomiting. Additionally, current therapies do not treat the underlying neurodegenerative disorder resulting in a continuing cognitive decline in patients.

### SUMMARY

Blocking of purine receptors, particularly adenosine receptors, and more particularly adenosine A_{2A} receptors may be beneficial in treatment or prevention of movement disorders such as Parkinson's disease, or disorders such as depression, cognitive, or memory impairment, acute and chronic pain, ADHD or narcolepsy, or for neuroprotection in a subject. One adenosine A_{2A} inhibitor is 3-(4-amino-3-methylbenzyl)-7-(furan-2-yl)-3H-[1,2,3]triazolo[4,5-d]pyrimidin-5-amine.

In one aspect, a composition includes crystal form B of 3-(4-amino-3-methylbenzyl)-7-(furan-2-yl)-3H-[1,2,3]triazolo[4,5-d]pyrimidin-5-amine (1). The composition can be substantially pure crystal form B of 1. The composition can be characterized by peaks in X-ray powder diffraction at 2θ of 7.64°, 10.70°, 12.23°, 21.46°, 22.25°, 22.79°, 24.25°, and 28.43° The composition can be characterized by peaks in X-ray powder diffraction at 2θ of 7.64°, 10.70°, 12.23°, 13.17°, 15.24°, 16.50°, 17.82°, 18.50°, 19.49°, 20.52°, 21.46°, 22.25°, 22.79°, 24.25°, 26.50°, 27.33°, and 28.43°. The composition can further include a pharmaceutically acceptable carrier.

In another aspect, a composition includes a solvate of 3-(4-amino-3-methylbenzyl)-7-(furan-2-yl)-3H-[1,2,3]triazolo[4,5-d]pyrimidin-5-amine. The composition can include a THF solvate, a methyl ethyl ketone solvate, a 1,4-dioxane solvate, or a 1,1,1,3,3,3-hexafluoropropan-2-ol solvate of 1. The solvate can be substantially pure. The solvate can be crystal form D of 1. The solvate can be crystal form E of 1. The solvate can be crystal form F of 1. The solvate can be crystal form G of 1. The solvate can be crystal form H of 1.

In another aspect, a method of preparing crystal form B of 1 includes contacting 1, an N-protected derivative thereof, or a combination thereof, with a sulfonic acid. The sulfonic acid can be methanesulfonic acid. Contacting with a sulfonic acid can include contacting with an aqueous solution of methanesulfonic acid having a concentration of 1 M or greater. The N-protected derivative of 1 can be 3-(4-trifluoroacetamido-3-methylbenzyl)-7-(furan-2-yl)-3H-[1,2,3]triazolo[4,5-d]pyrimidin-5-amine. The method can further include contacting 1, an N-protected derivative thereof, or a combination thereof, with a basic composition. The basic composition can be an aqueous potassium hydroxide solution. The concentration of potassium hydroxide in the aqueous potassium hydroxide solution can be greater than 1 M.

In another aspect, a method of preparing crystal form B of 3-(4-amino-3-methylbenzyl)-7-(furan-2-yl)-3H-[1,2,3]triazolo[4,5-d]pyrimidin-5-amine includes contacting 3-(4-amino-3-methylbenzyl)-7-(furan-2-yl)-3H-[1,2,3]triazolo[4,5-d]pyrimidin-5-amine with a carboxylic acid. The carboxylic acid can be formic acid, acetic acid, trichloroacetic acid, trifluoroacetic acid, propionic acid, butanoic acid, or a combination thereof. The method can further include contacting 3-(4-amino-3-methylbenzyl)-7-(furan-2-yl)-3H-[1,2,3]triazolo[4,5-d]pyrimidin-5-amine with a basic composition. The basic composition can be an aqueous ammonium hydroxide solution.

In another aspect, a method of making a compound includes combining in a vessel an amount of DADCP, an amount (3-methyl-4-nitrophenyl)methanamine hydrochloride with an amount of a sterically hindered amine and an amount of high boiling point alcohol, thereby forming a reaction mixture, and heating the reaction mixture to a temperature above 100° C. for a predetermined reaction time.

Heating the reaction mixture can include heating to a temperature of 120° C. or higher. The sterically hindered amine can be diisopropylethylamine (DIPEA), triisopropyl amine, triisobutyl amine, 2,4,6-collidine, 2,6-lutidine, 2,6-di-t-butylpyridine, or 1,4-diazabicyclo[2.2.2]ocatane. The high boiling point alcohol can be n-butanol, ethylene glycol, 1,4-butanediol, 1,3-butanediol, benzyl alcohol, t-amyl alcohol, n-pentanol, or 2-butoxyethanol. The method can including adding a diazotization reagent to the reaction mixture after the predetermined reaction time. The diazotization reagent can be a nitrite salt, such as sodium nitrite.

Other aspects, features, and objects will be apparent from the description and drawings.

### BRIEF DESCRIPTION OF THE DRAWINGS

FIGS. 1A-1C are graphs depicting properties of a crystalline form of a pharmaceutical.

FIGS. 2A-2C are graphs depicting properties of a crystalline form of a pharmaceutical.

FIGS. 3A-3C are graphs depicting properties of a crystalline form of a pharmaceutical.

FIGS. 4A-4C are graphs depicting properties of a crystalline form of a pharmaceutical.

FIGS. 5A-5B are graphs depicting properties of a crystalline form of a pharmaceutical.

FIGS. 6A-6D are graphs depicting properties of a crystalline form of a pharmaceutical.

FIGS. 7A-7B are graphs depicting properties of a crystalline form of a pharmaceutical.

FIG. 8 is a graph depicting properties of a crystalline form of a pharmaceutical.

FIG. 9 is a schematic depiction of a crystal structure of a pharmaceutical.

FIG. 10 is a schematic depiction of a crystal structure of a pharmaceutical.

### DETAILED DESCRIPTION

Blockade of A₂ adenosine receptors has been implicated in the treatment of movement disorders such as Parkinson's disease and in the treatment of cerebral ischemia. See, for example, Richardson, P. J. et al., Trends Pharmacol. Sci. 1997, 18, 338-344, and Gao, Y. and Phillis, J. W., Life Sci. 1994, 55, 61-65, each of which is incorporated by reference in its entirety.

Adenosine A_{2A} receptor antagonists have potential use in the treatment of movement disorders such as Parkinson's Disease (Mally, J. and Stone, T. W., CNS Drugs, 1998, 10, 311-320, which is incorporated by reference in its entirety).

Adenosine is a naturally occurring purine nucleoside which has a wide variety of well-documented regulatory functions and physiological effects. The central nervous system (CNS) effects of this endogenous nucleoside have attracted particular attention in drug discovery, because of the therapeutic potential of purinergic agents in CNS disorders (Jacobson, K. A. et al., J. Med. Chem 1992, 35, 407-422, and Bhagwhat, S. S.; Williams, M. E. Opin. Ther. Patents 1995, 5,547-558, each which is incorporated by reference in its entirety).

Adenosine receptors represent a subclass (P₁) of the group of purine nucleotide and nucleoside receptors known as purinoreceptors. The main pharmacologically distinct adenosine receptor subtypes are known as A₁, A_{2A}, A_{M} (of high and low affinity) and A₃ (Fredholm, B. B., et al., Pharmacol. Rev. 1994, 46, 143-156, which is incorporated by reference in its entirety). The adenosine receptors are present in the CNS (Fredholm, B. B., News Physiol. Sci., 1995, 10, 122-128, which is incorporated by reference in its entirety).

P₁ receptor-mediated agents can be useful in the treatment of cerebral ischemia or neurodegenerative disorders, such as Parkinson's disease (Jacobson, K. A., Suzuki, F., Drug Dev. Res., 1997, 39, 289-300; Baraldi, P. G. et al., Curr. Med. Chem. 1995, 2, 707-722; and Williams, M. and Bumnstock, G. Purinergic Approaches Exp. Ther. (1997), 3-26. Editor. Jacobson, Kenneth A.; Jarvis, Michael F. Publisher: Wiley-liss, New York, N.Y., which is incorporated by reference in its entirety).

It has been speculated that xanthine derivatives such as caffeine may offer a form of treatment for attention-deficit hyperactivity disorder (ADHD). A number of studies have demonstrated a beneficial effect of caffeine on controlling the symptoms of ADHD (Garfinkel, B. D. et al., Psychiatry, 1981, 26, 395-401, which is incorporated by reference in its entirety). Antagonism of adenosine receptors is thought to account for the majority of the behavioral effects of caffeine in humans and thus blockade of adenosine A_{2A} receptors may account for the observed effects of caffeine in ADHD patients. Therefore a selective adenosine A_{2A} receptor antagonist may provide an effective treatment for ADHD but with decreased side-effects.

Adenosine receptors can play an important role in regulation of sleep patterns, and indeed adenosine antagonists such as caffeine exert potent stimulant effects and can be used to prolong wakefulness (Porkka-Heiskanen, T. et al., Science, 1997, 276, 1265-1268, which is incorporated by reference in its entirety). Adenosine's sleep regulation can be mediated by the adenosine A_{2A} receptor (Satoh, S., et al., Proc. Natl. Acad. Sci., USA, 1996, 93: 5980-5984, which is incorporated by reference in its entirety). Thus, a selective adenosine A_{2A} receptor antagonist may be of benefit in counteracting excessive sleepiness in sleep disorders such as hypersomnia or narcolepsy.

Patients with major depression demonstrate a blunted response to adenosine agonist-induced stimulation in platelets, suggesting that a dysregulation of adenosine A_{2A} receptor function may occur during depression (Berk, M. et al., 2001, Eur. Neuropsycopharmacol. 11, 183-186, which is incorporated by reference in its entirety). Experimental evidence in animal models has shown that blockade of adenosine A_{2A} receptor function confers antidepressant activity (El Yacoubi, M et al., Br. J. Pharmacol. 2001, 134, 68-77, which is incorporated by reference in its entirety). Thus, adenosine A_{2A} receptor antagonists may be useful in treatment of major depression and other affective disorders in patients.

The pharmacology of adenosine A_{2A} receptors has been reviewed (Ongini, E.; Fredholm, B. B. Trends Pharmacol. Sci. 1996, 17(10), 364-372, which is incorporated by reference in its entirety). One possible mechanism in the treatment of movement disorders by adenosine A_{2A} antagonists is that A_{2A} receptors may be functionally linked dopamine D₂ receptors in the CNS. See, for example, Ferre, S. et al., Proc. Natl. Acad. Sci. USA 1991, 88, 7238-7241; Puxe, K. et al., Adenosine Adenine Nucleotides Mol. Biol. Integr. Physiol., (Proc. Int. Symp.), 5th (1995), 499-507. Editors: Belardinelr, Luiz; Pelleg, Amir. Publisher: KIuwer, Boston, Mass.; and Ferre, S. et al., Trends Neurosci. 1997, 20, 482-487, each of which is incorporated by reference in its entirety.

Interest in the role of adenosine A_{2A} receptors in the CNS, due in part to in vivo studies linking A_{2A} receptors with catalepsy (Ferre et al., Neurosci. Lett. 1991, 130, 1624; and Mandhane, S. N. et al., Eur. J. Pharmacol. 1997, 328, 135-141, each of which is incorporated by reference in its entirety), has prompted investigations into agents that selectively bind to adenosine A_{2A} receptors.

One advantage of adenosine A_{2A} antagonist therapy is that the underlying neurodegenerative disorder may also be treated. See, e.g., Ongini, E.; Adami, M.; Ferri, C.; Bertorelli, R., Ann. N.Y. Acad. Sci. 1997, 825(Neuroprotective Agents), 3048, which is incorporated by reference in its entirety. In particular, blockade of adenosine A_{2A} receptor function confers neuroprotection against MPTP-induced neurotoxicity in mice (Chen, J- F., J. Neurosci. 2001, 21, RC143, which is incorporated by reference in its entirety). In addition, consumption of dietary caffeine (a known adenosine A_{2A} receptor antagonist), is associated with a reduced risk of Parkinson's disease (Ascherio, A. et al, Ann. Neurol., 2001, 50, 56-63; and Ross G. W., et al., JAMA, 2000, 283, 2674-9, each of which is incorporated by reference in its entirety). Thus, adenosine A_{2A} receptor antagonists may confer neuroprotection in neurodegenerative diseases such as Parkinson's disease.

Xanthine derivatives have been disclosed as adenosine A_{2A} receptor antagonists for treating various diseases caused by hyperfunctioning of adenosine A₂ receptors, such as Parkinson's disease (see, for example, EP-A-565377, which is incorporated by reference in its entirety). One prominent xanthine-derived adenosine A_{2A} selective antagonist is CSC [8-(3-chlorostyryl)caffeine] (Jacobson et al., FEBS Lett., 1993, 323, 141-144, which is incorporated by reference in its entirety).

Theophylline (1,3-dimethylxanthine), a bronchodilator drug which is a mixed antagonist at adenosine A₁ and A_{2A} receptors, has been studied clinically. To determine whether a formulation of this adenosine receptor antagonist would be of value in Parkinson's disease an open trial was conducted on 15 Parkinsonian patients, treated for up to 12 weeks with a slow release oral theophylline preparation (150 mg/day), yielding serum theophylline levels of 4.44 mg/L after one week. The patients exhibited significant improvements in mean objective disability scores and 11 reported moderate or marked subjective improvement (Mally, J., Stone, T. W. J. Pharm. Pharmacol. 1994, 46, 515-517, which is incorporated by reference in its entirety).

KF 17837 [E-8-(3,4dimethoxystyryl)-1,3-dipropyl-7-methylxanthine] is a selective adenosine A_{2A} receptor antagonist which on oral administration significantly ameliorated the cataleptic responses induced by intracerebroventricular administration of an adenosine A_{2A} receptor agonist, CGS 21680. KF 17837 also reduced the catalepsy induced by haloperidol and reserpine. Moreover, KF 17837 potentiated the anticataleptic effects of a subthreshold dose of L-DOPA plus benserazide, suggesting that KF 17837 is a centrally active adenosine A_{2A} receptor antagonist and that the dopaminergic function of the nigrostriatal pathway is potentiated by adenosine A_{2A} receptor antagonists (Kanda, T. et al., Eur. J. Pharmacol. 1994, 256, 263-268, which is incorporated by reference in its entirety). The structure activity relationship (SAR) of KF 17837 has been published (Shimada, J. et al., Bioorg. Med. Chem. Lett. 1997, 7, 2349-2352, which is incorporated by reference in its entirety). Recent data has also been provided on the adenosine A_{2A} receptor antagonist KW-6002 (Kuwana, Y et al., Soc. Neurosci. Abstr. 1997,23, 119.14; and Kanda, T. et al., Ann. Neurol. 1998,43(4), 507-513, each of which is incorporated by reference in its entirety).

Non-xanthine structures sharing these pharmacological properties include SCH 58261 and its derivatives (Baraldi, P. G. et al., J. Med Chem. 1996, 39, 1164-71, which is incorporated by reference in its entirety). SCH 58261 (7-(2-phenylethyl)-5-amino-2-(2-furyl)-pyrazolo-[4,3-e]-1,2,4triazolo[1,5-c]pyrimidine) is reported as effective in the treatment of movement disorders (Ongini, E. Drug Dev. Res. 1997, 42(2), 63-70, which is incorporated by reference in its entirety) and has been followed up by a later series of compounds (Baraldi, P. G. et al., J. Med. Chem. 1998,41(12), 2126-2133, which is incorporated by reference in its entirety).

One adenosine A_{2A} inhibitor is 3-(4-amino-3-methylbenzyl)-7-(furan-2-yl)-3H-[1,2,3]triazolo[4,5-d]pyrimidin-5-amine (1). See International Patent Application Publication WO 02/055083, which is incorporated by reference in its entirety.

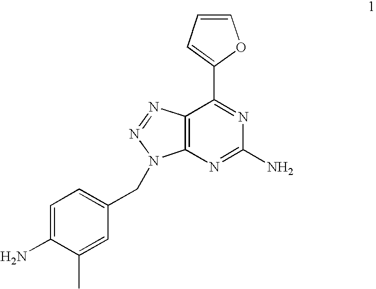

Compound 1 can be synthesized using any conventional technique, several of which are exemplified below. Preparation of 1 is described generally in WO 02/055083 (see, e.g., pages 23-28, 42, 66-67, and 106).

More particularly, WO 02/055083 describes the following sequence of reactions:

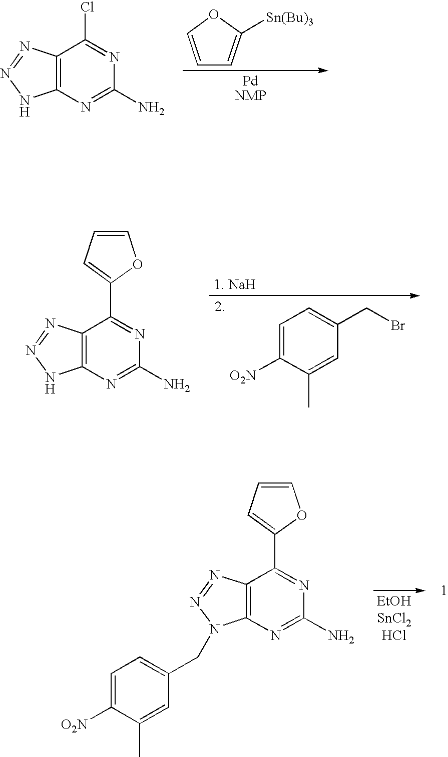

The melting point reported for compound 1 prepared by the above method was 245.3° C.-246.1° C. (see page 106). As discussed further below, this melting point is characteristic of crystal form A.

In one embodiment, synthesis of compound 1 relies on the reaction of a tosylated pyrimidine 2 with 3-methyl-4-triflouroacetamido-benzylamine 3. The final step in this route is removal of the trifluoroacetyl protecting group by basic hydrolysis.

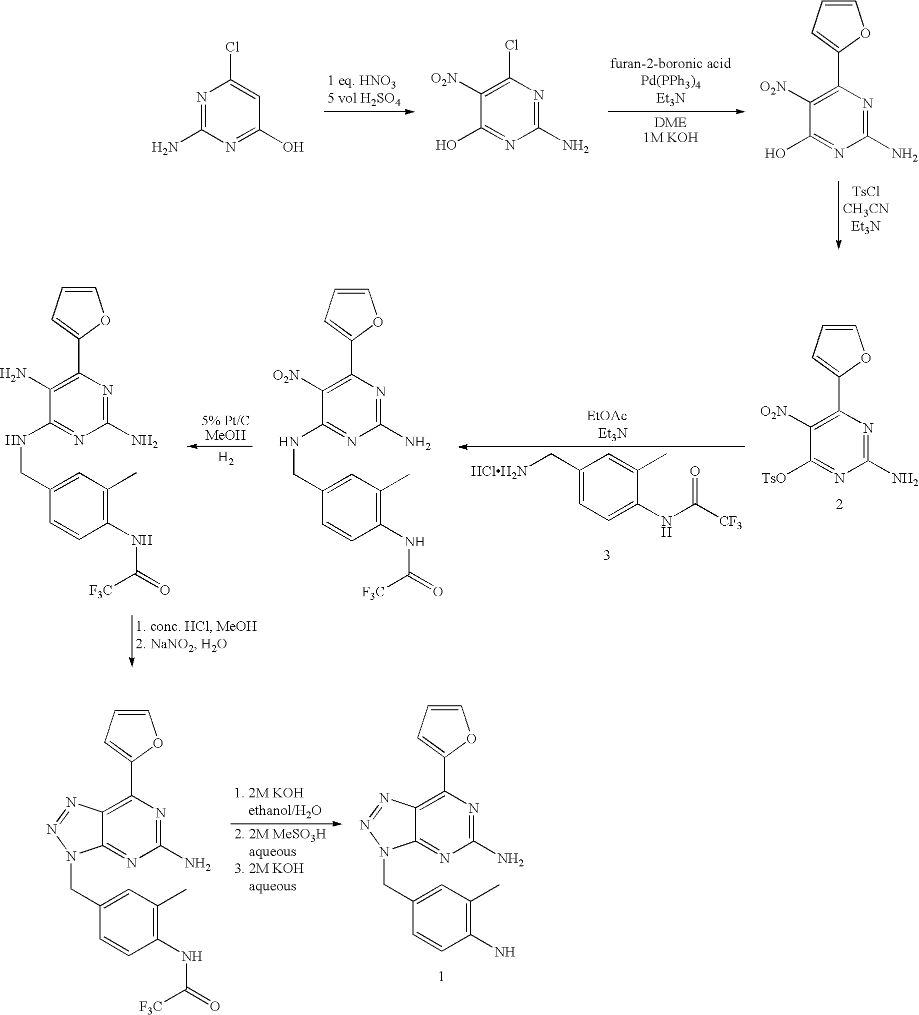

When prepared by the method above, crystal form B of compound 1 was obtained. In some circumstances, the 4-aminobenzyl group can be protected with by a methylcarbonyloxy or benzylcarbonyloxy protecting group, instead of a trifluoroacetyl protecting group.

In other embodiments, synthesis of compound 1 proceeds by forming the triazole ring prior to forming the pyrimidine ring, as illustrated in the schemes below.

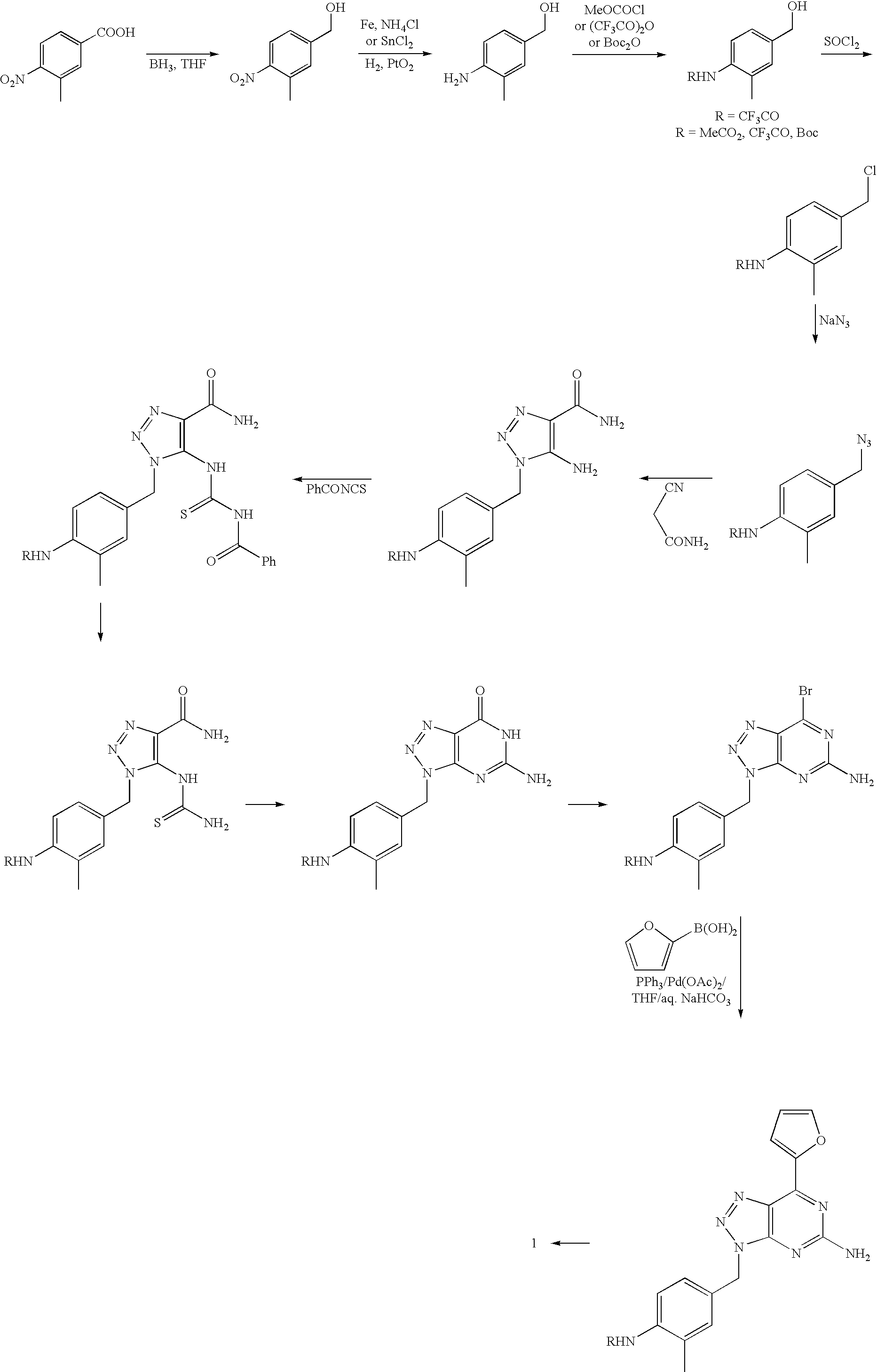

Another route that builds the triazole ring before the pyrimidine ring is:

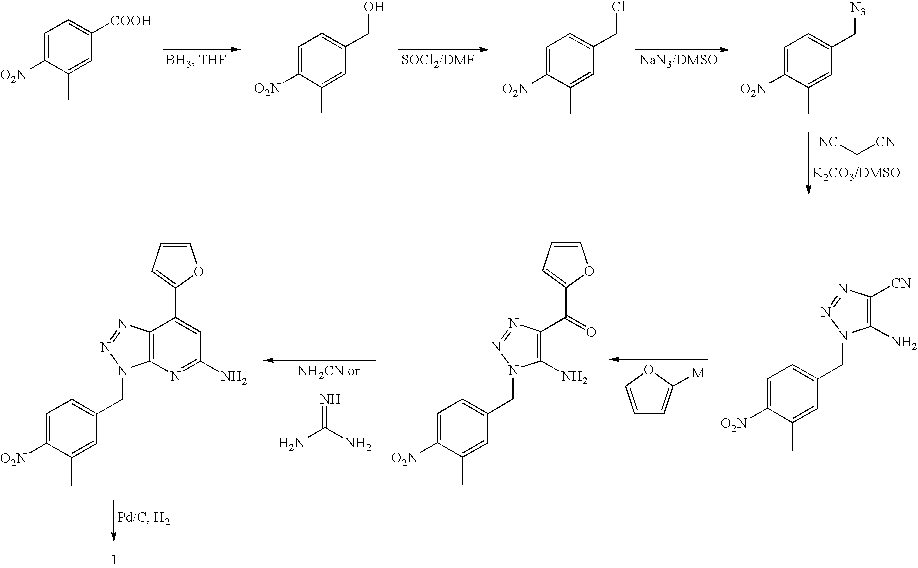

In another embodiment, synthesis of compound 1 involves the reaction of the pyrimidine 4 with a diazonium species:

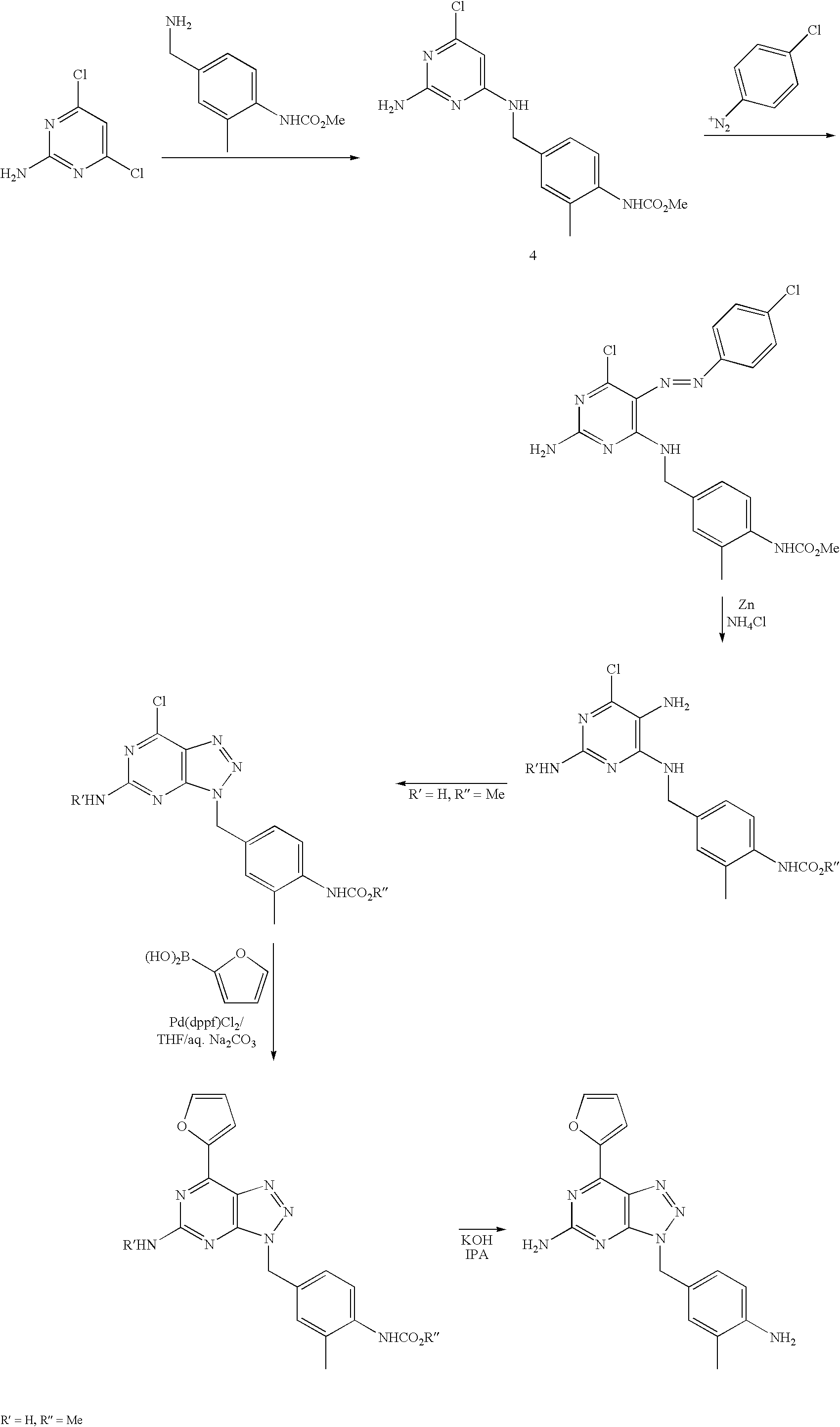

In yet another embodiment, the synthetic method can involve the coupling of N-(2-amino-4,6-dichloropyrimidin-5-yl)-formamide with 3-methyl-4-nitrobenzamide.

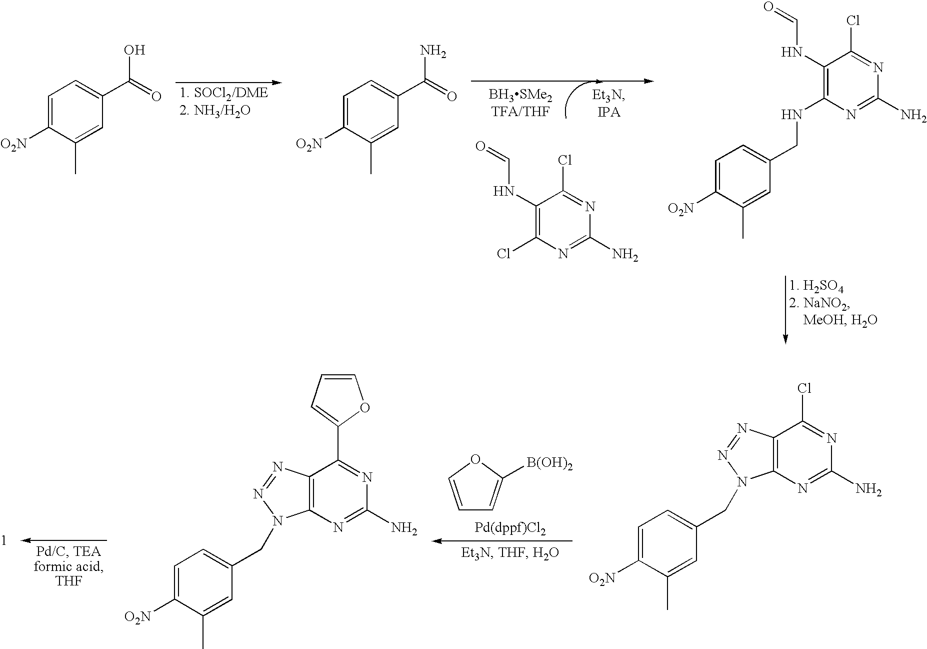

A variation of the above method, in which the coupling and diazotization steps can be carried out in one pot, without separation, can also be used.

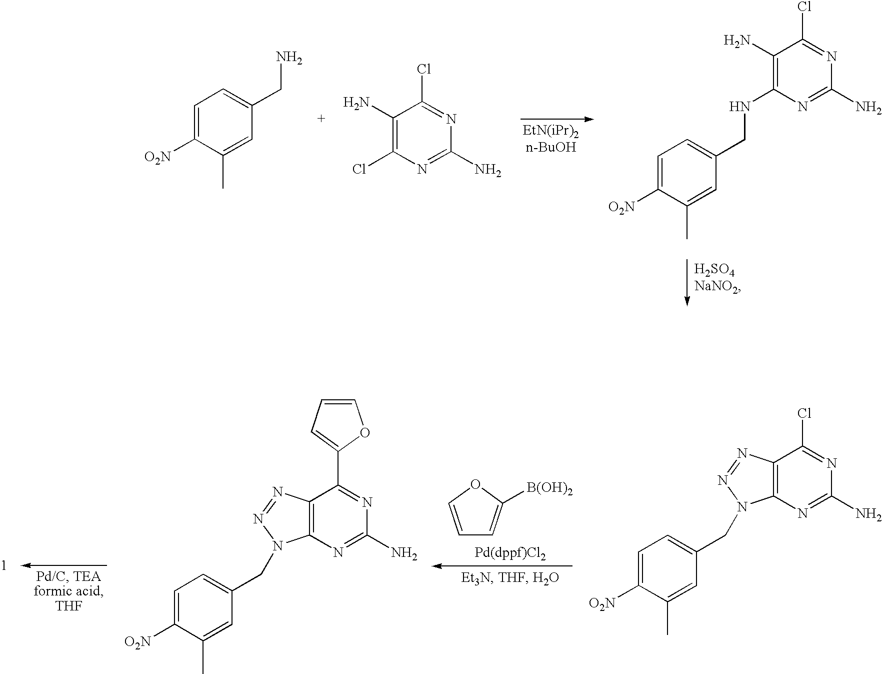

In this method, the coupling reaction can be favored by using a sterically hindered amine and a high-boiling point alcohol as a solvent. The sterically hindered amine is preferably to substantially basic and substantially non-nucleophilic. Some examples of suitable sterically hindered amines include diisopropylethylamine (DIPEA), triisopropyl amine, triisobutyl amine, 2,4,6-collidine, 2,6-lutidine, 2,6-di-t-butylpyridine, and 1,4-diazabicyclo[2.2.2]ocatane. In some embodiments, a sterically hindered amine can be more sterically hindered than triethylamine. The high-boiling point alcohol can have a boiling point higher than that of water (i.e., 100° C. at atmospheric pressure). Some examples of suitable high-boiling point alcohols are n-butanol, ethylene glycol, 1,4-butanediol, 1,3-butanediol, benzyl alcohol, t-amyl alcohol, n-pentanol, and 2-butoxyethanol. The product of the coupling reaction can be combined with a diazotization reagent (e.g., NaNO₂) in the same pot, without the need to isolate the product of the coupling reaction. Thus, a straightforward, one-pot synthesis of an important intermediate is provided.

Compound 1 can exist in a variety of crystal forms, distinguished by, for example, X-ray powder diffraction patterns, DSC measurements, and solvent content. The various crystal forms are designated Form A, Form B, Form D, Form E, Form F, Form G, and Form H.

Form A can be prepared by dissolving compound 1 in a suitable solvent, such as tetrahydrofuran (THF), N,N-dimethylformamide (DMF), N,N-dimethylacetamide (DMA), N-methylpyrrolidone (NMP), or a mixture thereof at a temperature suitable for dissolution of compound 1. Alternatively, compound 1 can be dissolved in a mixture of a solvent, (e.g., THF, DMF, DMA, or NMP) and an antisolvent, such as water, methanol, ethanol, isopropyl alcohol, n-butyl alcohol, t-butyl methyl ether (TBME), acetone, acetonitrile, 1,2-dimethoxyethane, or a mixture thereof, at a temperature suitable for dissolution of compound 1. An antisolvent can then be added to the mixture under conditions suitable for the formation of Form A. For example, compound 1 can be dissolved in DMSO and then combined with an alcohol, for example, methanol, ethanol, propanol, isopropanol, n-butyl alcohol, sec-butyl alcohol, or t-butyl alcohol, and, optionally, with a second anti-solvent such as an alcohol or water.

Form A can also be prepared by dissolving compound 1 in a mixture of a solvent and an acid. Some suitable solvents for this method include THF, ethanol, and methanol. Some suitable acids include hydrochloric acid, sulfuric acid, and methanesulfonic acid. Once dissolved in the solvent/acid mixture, compound 1 is then precipitated by addition a suitable base, such as a hydroxide or an amine, (for example, aqueous sodium hydroxide) under conditions suitable for the production of Form A.

Form B can be prepared by dissolving compound 1 in a mixture of a solvent and an acid, particularly water and methanesulfonic acid, and precipitating compound 1 by addition a suitable base, such as a hydroxide, or an amine, (e.g., aqueous potassium hydroxide) under conditions suitable for the production of Form B. For example, crystal form B can be prepared by dissolving compound 1 (or a protected form, e.g., a form in which the phenyl amino group is acylated, such as with an acetyl or trifluoroacetyl group) in a solution of water and an alkyl sulfonic acid, such as methanesulfonic acid or ethanesulfonic acid, and adding an organic solvent, such as ethyl acetate (for example, to extract any remaining protected 1), and a base, such as a hydroxide base like sodium hydroxide, potassium hydroxide, or ammonium hydroxide. Addition of the base can result in precipitation of 1. The precipitate can be reslurry (e.g., in water or an aqueous solvent system) to remove any residual alkyl sulfonic acid.

Alternatively, crystal form B can be forming a slurry of compound 1 in a mixture of water and an alkyl acid, such as, for example, formic acid, acetic acid, trichloroacetic acid, trifluoroacetic acid, propionic acid, butanoic acid, or the like, and neutralizing the mixture with a base, such as a hydroxide base like sodium hydroxide, potassium hydroxide, or ammonium hydroxide.

The compound can be used in the form of pharmaceutically acceptable salts derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The compound may be formulated into pharmaceutical compositions that may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutical compositions can include compound 1, or pharmaceutically acceptable derivatives thereof, together with any pharmaceutically acceptable carrier. The term “carrier” as used herein includes acceptable adjuvants and vehicles. Pharmaceutically acceptable carriers that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as do natural pharmaceutically-acceptable oils, such as olive oil or castor oil, especially in their polyoxyethylated versions. These oil solutions or suspensions may also contain a long-chain alcohol diluent or dispersant.

The pharmaceutical compositions can be orally administered in any orally acceptable dosage form including, but not limited to, capsules, tablets, aqueous suspensions or solutions.

In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions may be administered in the form of suppositories for rectal administration. These can be prepared by mixing the agent with a suitable non-irritating excipient which is solid at room temperature but liquid at the rectal temperature and therefore will melt in the rectum to release the drug. Such materials include cocoa butter, beeswax and polyethylene glycols.

The pharmaceutical compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient that may be combined with the carrier materials to produce a single dosage form will vary depending upon the host treated, and the particular mode of administration. It should be understood, however, that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredient may also depend upon the therapeutic or prophylactic agent, if any, with which the ingredient is co-administered.

A pharmaceutical composition can include an effective amount of compound 1. An effective amount is defined as the amount which is required to confer a therapeutic effect on the treated patient, and will depend on a variety of factors, such as the nature of the inhibitor, the size of the patient, the goal of the treatment, the nature of the pathology to be treated, the specific pharmaceutical composition used, and the judgment of the treating physician. For reference, see Freireich et al., Cancer Chemother. Rep. 1966, 50, 219 and Scientific Tables, Geigy Pharmaceuticals, Ardley, N.Y., 1970, 537. Dosage levels of between about 0.001 and about 100 mg/kg body weight per day, preferably between about 0.1 and about 10 mg/kg body weight per day of the active ingredient compound are useful.

The following examples are for the purpose of illustration only and are not intended to be limiting.

### EXAMPLES

### Example 1

### Preparation of N-[2-amino-4-chloro-6-(3-methyl-4-nitro-benzylamino)-pyrimidin-5-yl]formamide

Isopropanol (1500 mL), N-(2-amino-4,6-dichloro-pyrimidin-5-yl)-formamide (100.0 g) and (3-methyl-4-nitrophenyl)methanamine hydrochloride (263.47 g) were charged to a 5 L reactor. The temperature was increased to 58-65° C., and triethylamine (341.85 mL) was added with vigorous stirring over a period of 30-40 min. The reaction mixture was heated to reflux for 3-4 hr. Reaction mass temperature was brought down to 15-20° C., water (2000 mL) was added over a period of 30 min. Stirring was continued at 15-20° C. for another 1-2 hr. The reaction mass was filtered and washed with an isopropyl alcohol/water mixture (140 mL/180 mL) followed by water (215.0 mL) and cold isopropyl alcohol (95.0 mL). The product was dried at 40-45° C. for 10-15 hr under vacuum to yield 150-155 g (92-95%) of N-[2-amino-4-chloro-6-(3-methyl-4-nitro-benzylamino)-pyrimidin-5-yl]-formamide.

### Example 2

### Preparation of 7-chloro-3-(3-methyl-4-nitrobenzyl)-3H-[1,2,3]triazolo[4,5-d]pyrimidin-5-amine

To a three-neck round-bottomed flask equipped with a reflux condenser, a thermometer, a mechanical stirrer and a nitrogen inlet was added methanol (70.0 mL), sulfuric acid (4.51 mL, 84.6 mmol) and N-[2-amino-4-chloro-6-(3-methyl-4-nitro-benzylamino)-pyrimidin-5-yl]-formamide (10.2 g, 28.8 mmol) at room temperature. The resultant clear solution was heated to 60° C. over 10 min and 20 mL of solvent was collected under vacuum distillation at 50 to 60° C. over 20 min. The reaction was cooled to room temperature and water (150 mL) was added to give bright yellow slurry.

To the slurry was added sodium nitrite (40 wt % aqueous solution, 4.80 mL, 36.0 mmol) over 4 hours at room temperature. The resultant thick slurry was aged for an additional hour before filtration. The wet cake was washed with water (50 mL), ammonium hydroxide (0.5 N, 50 mL) and then water (50 mL). The crude product was dried under vacuum to constant weight to yield 9.25 g (99.6%) of 7-chloro-3-(3-methyl-4-nitrobenzyl)-3H-[1,2,3]triazolo]4,5-d]pyrimidin-5-amine.

### Example 2A

### Alternate Preparation of 7-chloro-3-(3-methyl-4-nitrobenzyl)-3H-[1,2,3]triazolo[4,5-d]pyrimidin-5-amine

2,5-Diamino-4,6-dichloropyrimidine (DADCP) (19.6 g, 109 mmol, 1.00 eq), (3-methyl-4-nitrophenyl)methanamine hydrochloride (19.9 g, 98.2 mmol, 0.90 eq), 1-butanol (300 mL) and diisopropylethylamine (DIPEA, 43.0 mL, 260 mmol, 2.4 eq) were mixed in a 750 mL reaction vessel and heated to 120° C. After 3 to 3.5 hours at that temperature, the reaction mixture was cooled to room temperature. An additional portion of (3-methyl-4-nitrophenyl)methanamine hydrochloride (5.50 g, 0.25 eq) was added. The reaction mixture was heated again to 120° C. for an additional 3 to 4 hours, then cooled again to room temperature.

Methanol (100 mL) was added at 18° C., followed by potable water (30 mL). Concentrated sulfuric acid (13.0 g, 132 mmol, 1.2 eq) was added in 5-10 minutes, and the solution was cooled to 20° C. A solution of NaNO₂ (8.30 g 119 mmol, 1.1 eq) in 30 mL of potable water was added in 20-30 minutes, maintaining a temperature between 20 and 25° C. After addition, the reaction suspension was stirred for 1-2 hours at 17-19° C. The mixture was filtered and washed with 75 mL of methanol, 75 mL of 0.1N ammonia solution, and 75 mL of water. After vacuum drying at 80° C., 25.7 g of 7-chloro-3-(3-methyl-4-nitrobenzyl)-3H-[1,2,3]triazole[4,5-d]pyrimidin-5-amine (73.4% yield) was obtained.

### Example 3

### Preparation of 7-(furan-2-yl)-3-(3-methyl-4-nitrobenzyl)-3H-[1,2,3]triazolo[4,5-d]pyrimidin-5-amine

A 1 L reaction vessel was charged with 7-chloro-3-(3-methyl-4-nitrobenzyl)-3H-[1,2,3]triazolo[4,5-d]pyrimidin-5-amine (50.0 g, 156.4 mmol), and Pd(dppf)Cl₂ (185 mg, 0.234 mmol). The vessel was then evacuated and flushed with nitrogen 4 times to remove oxygen. Next, triethylamine (65.4 mL, 469 mmol), degassed water (300 mL) and degassed THF (200 mL) was added via cannula. The slurried material was then heated to 68° C. and held at that temperature for 15 minutes. In a 500 mL Schott bottle, equipped as described above, was charged 2-furylboronic acid (21.0 g, 188 mmol). The bottle was flushed with nitrogen and degassed THF (200 mL) was pumped in. After all the boronic acid had dissolved, the solution was added to the 1 L reaction vessel with a pump over the course of 1 hour. The reaction temperature was maintained at 68° C. during the addition. The reaction was allowed to stir at 68° C. for an additional 3 hours (total reaction time was 4 hours), and then the reaction was cooled to 25° C. The final product, off-white crystals, was collected by filtration. The filter cake was washed with methanol (400 mL in two parts) to remove any colored impurities. The product was dried under vacuum to yield 45.3 g (82%) of 7-(furan-2-yl)-3-(3-methyl-4-nitrobenzyl)-3H-[1,2,3]triazolo[4,5-d]pyrimidin-5-amine.

### Example 4

### Preparation of 3-(4-amino-3-methylbenzyl)-7-(furan-2-yl)-3H-[1,2,3]triazolo[4,5-d]pyrimidin-5-amine (1)

A 250 mL 2-necked round-bottomed flask was charged with 7-(furan-2-yl)-3-(3-methyl-4-nitrobenzyl)-3H-[1,2,3]triazolo[4,5-d]pyrimidin-5-amine (3.0 g, 8.5 mmol) and 5% Pd/C catalyst (0.46 g, 0.073 mmol) under a nitrogen atmosphere. Next, THF (72 mL) and triethylamine (9.0 mL, 65 mmol) were added via syringe and the resulting mixture was stirred to obtain slurry. Formic acid (2.3 mL, 46.03 mmol) was then added all at once, and the mixture was heated with a bath set to a temperature of 70° C. After 5 hours the reaction was cooled to 25° C. Water (60 mL) was added, and concentrated hydrochloric acid was added dropwise to dissolve the product. The solution was filtered through Celite 545 to remove the catalyst, and the filter cake was washed with additional water (2×5 mL). To the yellowish filtrate was added 50% sodium hydroxide in water to precipitate the product. The mixture was stirred for an additional hour before isolation by filtration. The filter cake was washed with water (10 mL) then methanol (10 mL). The product was dried under vacuum to constant weight to yield 2.79 g (92%) of 3-(4-amino-3-methylbenzyl)-7-(furan-2-yl)-3H-[1,2,3]triazolo[4,5-d]pyrimidin-5-amine.

### Example 5

### Characterization of Crystal Form A of 1

A sample of 1 in crystal form A was prepared by charging a 250 mL round bottom flask with compound 1 (10.0 g) and DMSO (45 mL) at room temperature. The resultant slurry was heated to 75° C. to give a clear solution. Isopropanol (90 mL) was added to the solution over 2 hours at 75° C. and then cooled to room temperature. The mixture was filtered at room temperature and washed with a DMSO/isopropanol mixture (13 mL/26 mL) followed by isopropanol (40 mL). The product was dried under vacuum to yield 9.59 g (95.9%) of the crystal form A of 1. The sample was characterized by X-ray powder diffraction (XRPD), differential scanning calorimetry (DSC), and thermogravimetric analysis (TGA).

Alternatively, a glass lined 1000 L reactor was charged with 58.3 kg wet, crude compound 1. After purging the reactor with nitrogen, the reactor was charged with 289 kg DMSO, and the mixture was heated to 77-83° C. A solution was obtained, to which 210 L of ethanol 96% was added in 75 min at 77-83° C., whereby crystallization started. Then, 105 L of purified water were added in 45 min at 77-83° C. After the addition of water was complete, the mixture was cooled to 20-25° C. in 3 hours and stirred at this temperature for 1 hour. The product was filtered, and the filter cake was washed three times with 84 L of ethanol 96% each, the first two washings being performed with stirring. Finally, the product, wet, pure compound 1, was discharged.

For XRPD, the relative intensities of the peaks can vary depending on, for example, the sample preparation technique, the sample mounting procedure, and the particular instrument employed. Instrument variation and other factors can also affect the measured values of 2θ. Accordingly, XRPD peak assignments can vary by plus or minus 0.2° in 2θ.

For DSC, observed temperatures will depend on the rate of temperature change as well as sample preparation technique and the particular instrument employed. Thus, the values reported for DSC thermograms can vary by plus or minus about 4° C.

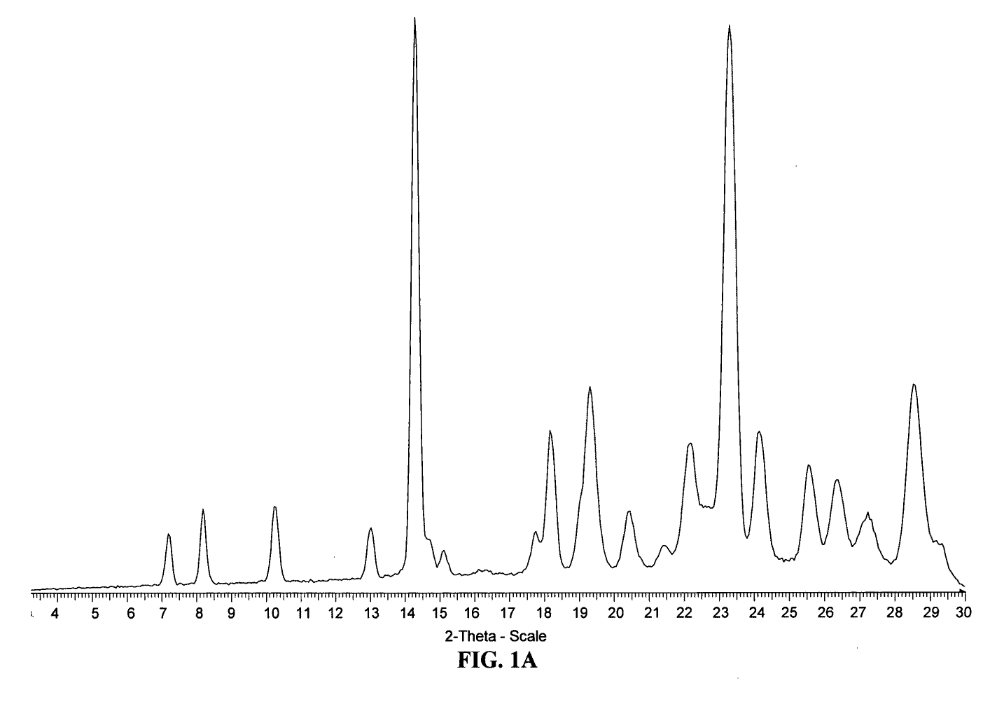

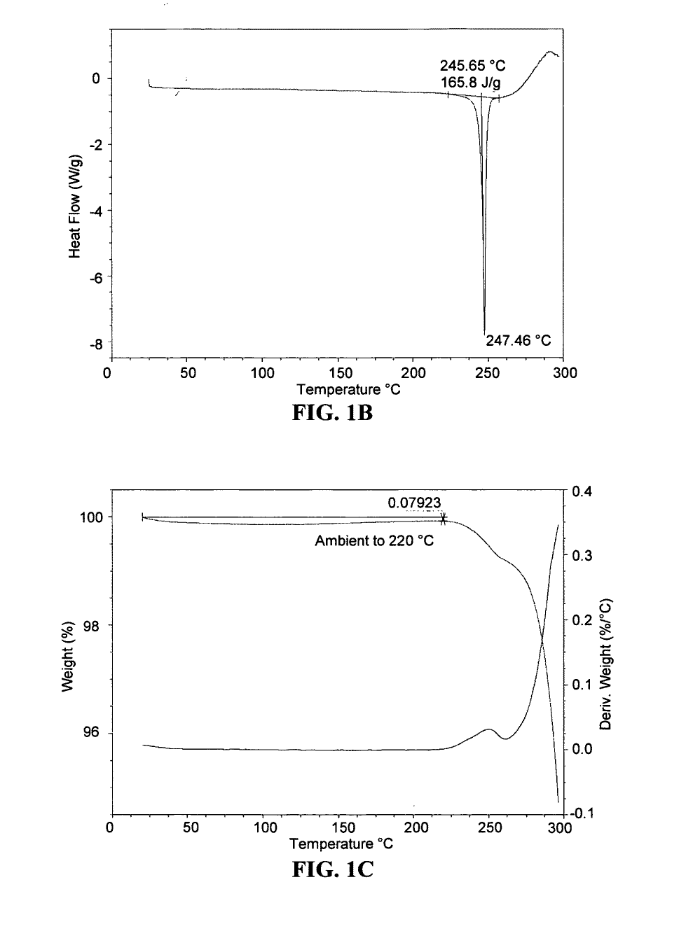

FIG. 1A shows an XRPD trace of crystal form A. The XRPD pattern of crystal form A is characterized by peaks at 2θ of 7.20°, 8.14°, 10.26°, 13.00°, 14.23°, 15.10°, 17.75°, 18.20°, 19.31°, 20.41°, 22.15°, 23.36°, 24.19°, 25.55°, 26.39°, 27.26°, and 28.62°.

FIG. 1B shows a DSC thermogram for crystal form A. Crystal form A shows a minimum in DSC thermograms (i.e., melting point) at about 243° C.-246° C., with a ΔH_{f} of between 154.5 J/g and 165.8 J/g. DSC analysis before and after micronisation showed no significant difference in the heat of fusion, confirming that micronisation did not adversely affect crystalline quality.

FIG. 1C shows a TGA trace for crystal form A. TGA revealed that form A was substantially free of solvent; weight loss from ambient temperature to 220° C. varied between <0.1% w/w to 1.2% w/w. TGA analysis before and after micronisation showed that micronised material contained less unbound and less trapped solvent than the pre-micronised material.

### Example 6

### Characterization of Crystal Form B of 1

A sample of 1 in crystal form B was prepared by charging MeSO₃H (143 mL), H₂O (1000 mL) and compound 1 to a clean flask and agitating for 15 min. Compound 1 dissolved in the MeSO₃H solution. If all of the mixture did not dissolve, it was heated to 30° C. to give complete dissolution. The vessel was charged with EtOAc (500 mL) and agitated for a further 30 min. The EtOAc layer was removed and the acidic reaction mixture was neutralized to pH 7 with 2M KOH aqueous solution. A light brown precipitate formed. The mixture was filtered, washed with H₂O (1000 mL) and dried in a vacuum oven at 50° C. to constant weight yielding compound 1 in crystal form B. If ¹H NMR indicated the presence of potassium methanesulfonate, it was removed by a slurry in H₂O (20 volumes).

Alternatively, crystal form B was prepared by charging a 100 mL round bottom flask with compound 1 (5.17 g), acetic acid (20 mL) and water (30 mL) at room temperature. The resultant slurry was stirred at room temperature for 6 h. The mixture was filtered and washed with 0.5 N ammonium hydroxide followed by water. The product was dried under vacuum to yield 5.00 g (96.7%) of the crystal form B of 1.

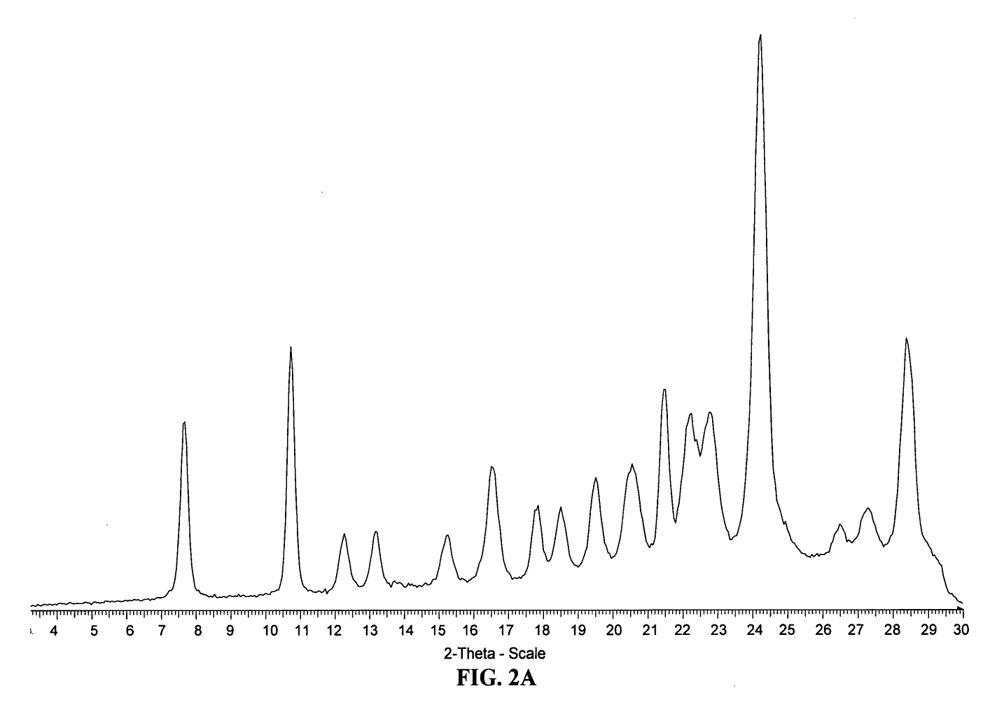

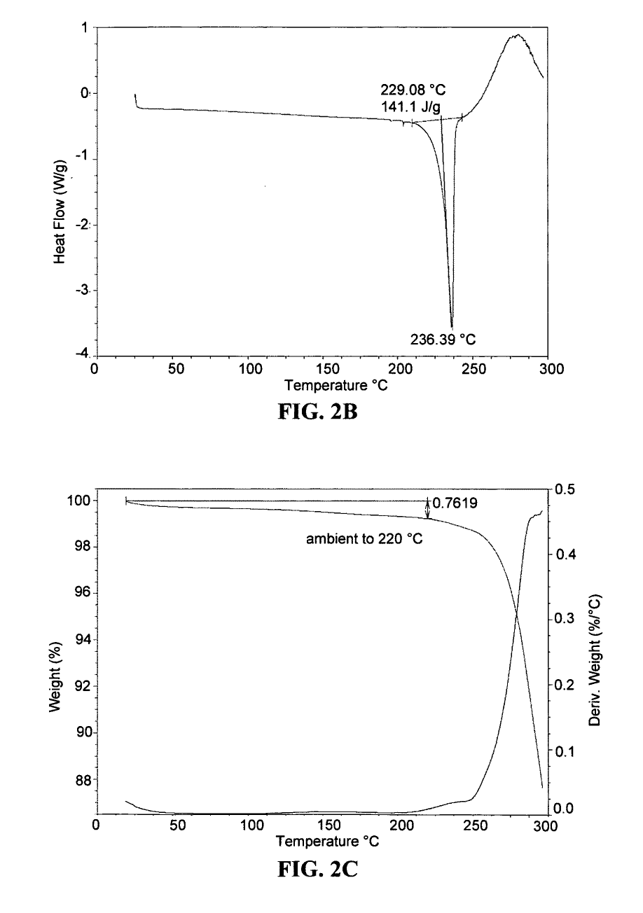

Crystal form B was characterized by XRPD, DSC, and TGA. FIG. 2A shows an XRPD trace of crystal form B. The XRPD pattern of crystal form B is characterized by peaks at 2θ of 7.64°, 10.70°, 12.23°, 13.17°, 15.24°, 16.50°, 17.82°, 18.50°, 19.49°, 20.52°, 21.46°, 22.25°, 22.79°, 24.25°, 26.50°, 27.33°, and 28.43°. FIG. 2B shows a DSC thermogram for crystal from B. Crystal form B shows a minimum in DSC thermograms (i.e., melting point) at about 229° C., with a ΔH_{f} of 141.1 J/g. FIG. 2C shows a TGA trace for crystal form B. TGA revealed that form B was substantially free of solvent; weight loss from ambient temperature to 220° C. was 0.8% w/w.

### Example 7

### Characterization of Crystal Form D of 1 (THF solvate)

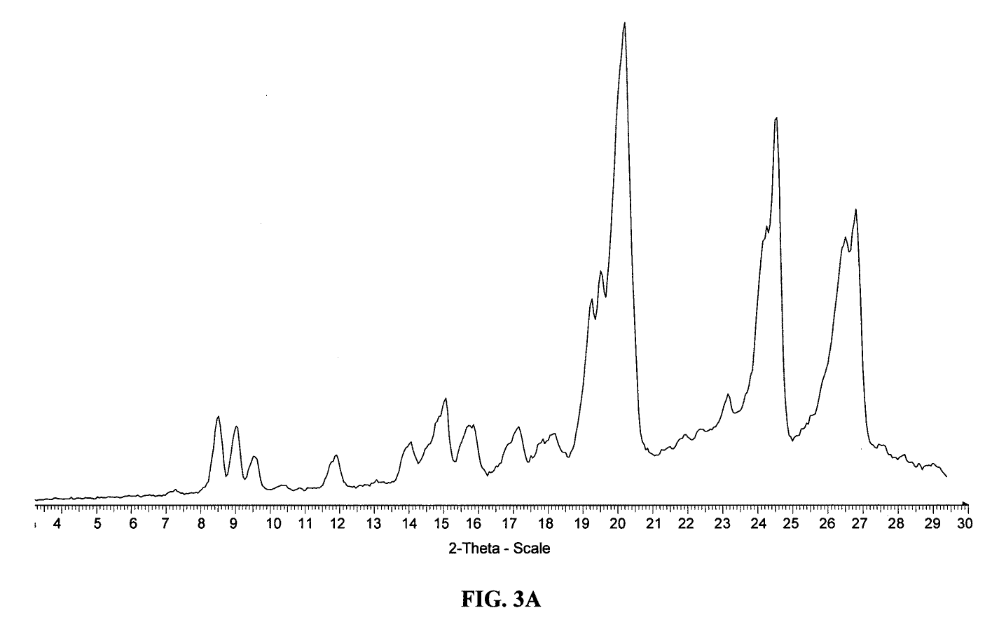

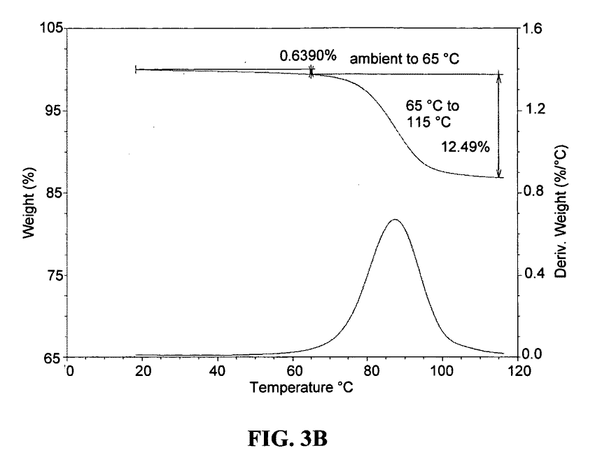

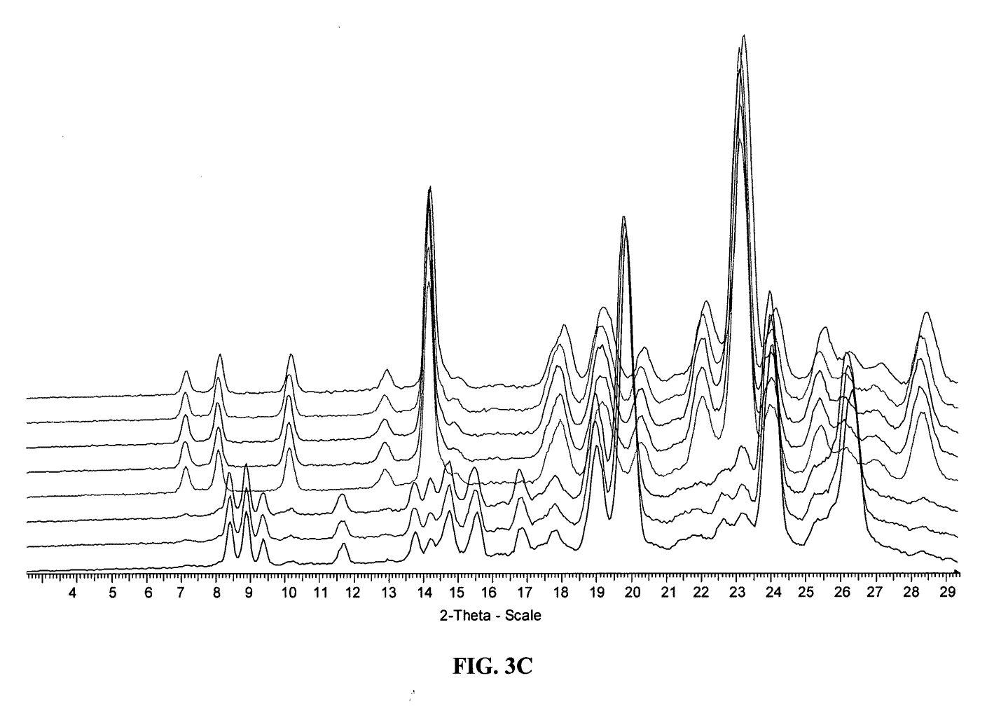

A sample of 1 in crystal form D was prepared by recystallization from hot THF. The sample was characterized by XRPD and TGA. FIG. 3A shows the XRPD trace of Form D, which is characterized by peaks at 2θ of 8.49°, 9.00°, 9.55°, 11.85°, 14.04°, 15.01°, 15.78°, 17.13°, 18.13°, 19.21°, 19.50°, 20.20°, 23.14°, 24.23°, 24,51°, 26.26°, and 26.81°. FIG. 3B shows a TGA trace for crystal form D. TGA revealed that form D lost 12.5% of its weight upon heating from ambient temperature to 65° C., or 0.7 moles solvent per mole of 1, consistent with form D being a THF hemisolvate.

FIG. 3C shows variable temperature XRPD traces of crystal form D. Traces (from bottom to top) were recorded at ambient temperature, 50° C., 65° C., 115° C., 140° C., 170° C., after cooling to 140° C., and after cooling to 30° C. The final product was crystal form A.

### Example 8

### Characterization of Crystal Form E of 1 (1,4-dioxane solvate)

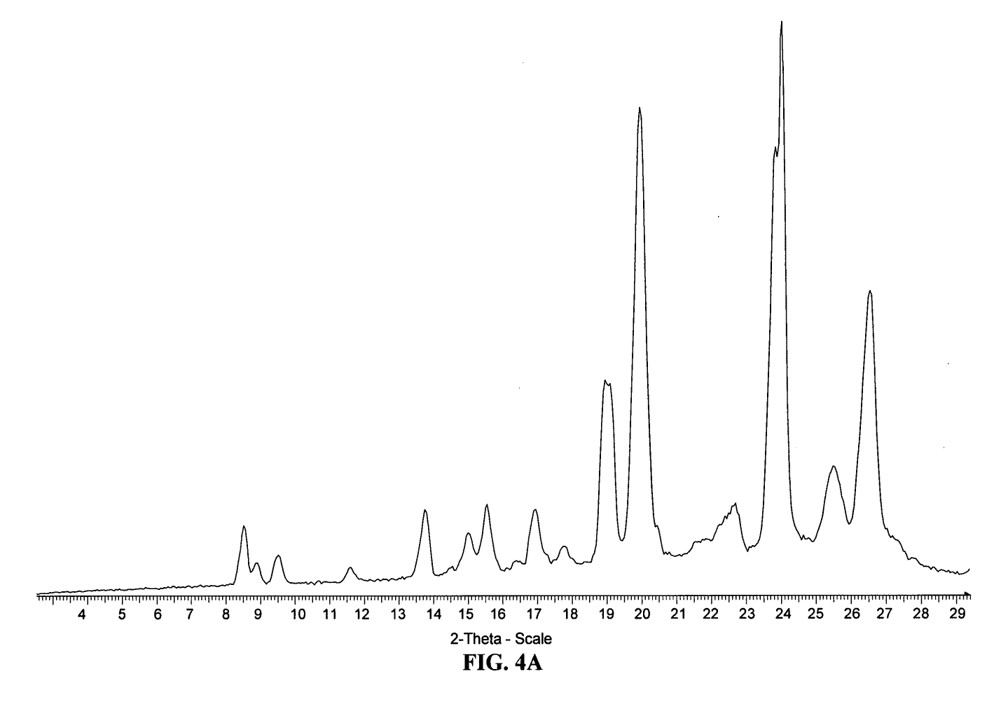

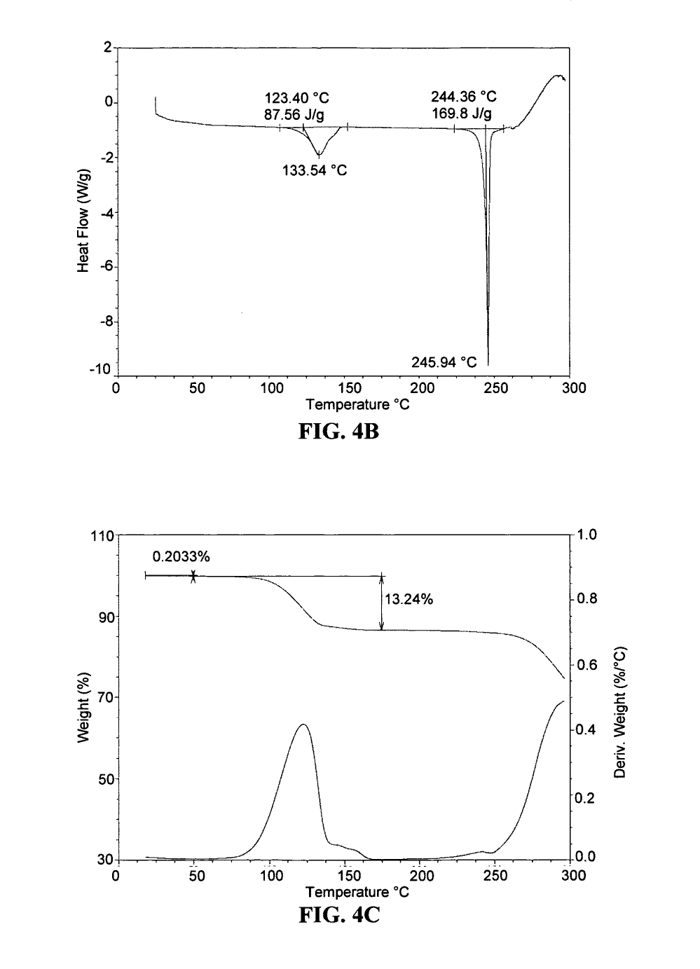

A sample of 1 in crystal form E was prepared by recrystallization from 1,4-dioxane. The sample was characterized by XRPD, DSC, and TGA. FIG. 4A shows an XRPD trace of crystal form E. The XRPD pattern of crystal form E is characterized by peaks at 2θ of 8.49°, 8.84°, 9.50°, 11.60°, 13.73°, 14.99°, 15.56°, 16.95°, 17.77°, 19.03°, 19.96°, 22.70°, 23.83°, 24.05°, 25.51°, and 26.57°. FIG. 4B shows a DSC thermogram for crystal form E. DSC thermograms of crystal form E show a desolvation endotherm above 123° C., and a minimum in (i.e., melting point) at about 244° C. The melting point of desolvated form E suggests that form E converts to form A upon desolvation.

FIG. 4C shows a TGA trace for crystal form E. TGA revealed that form E lost 13% of its weight upon heating from ambient temperature to 50° C., or 0.6 moles solvent per mole of 1, consistent with form D being a 1,4-dioxane hemisolvate.

### Example 9

### Characterization of Crystal Form F of 1 (methyl ethyl ketone solvate)

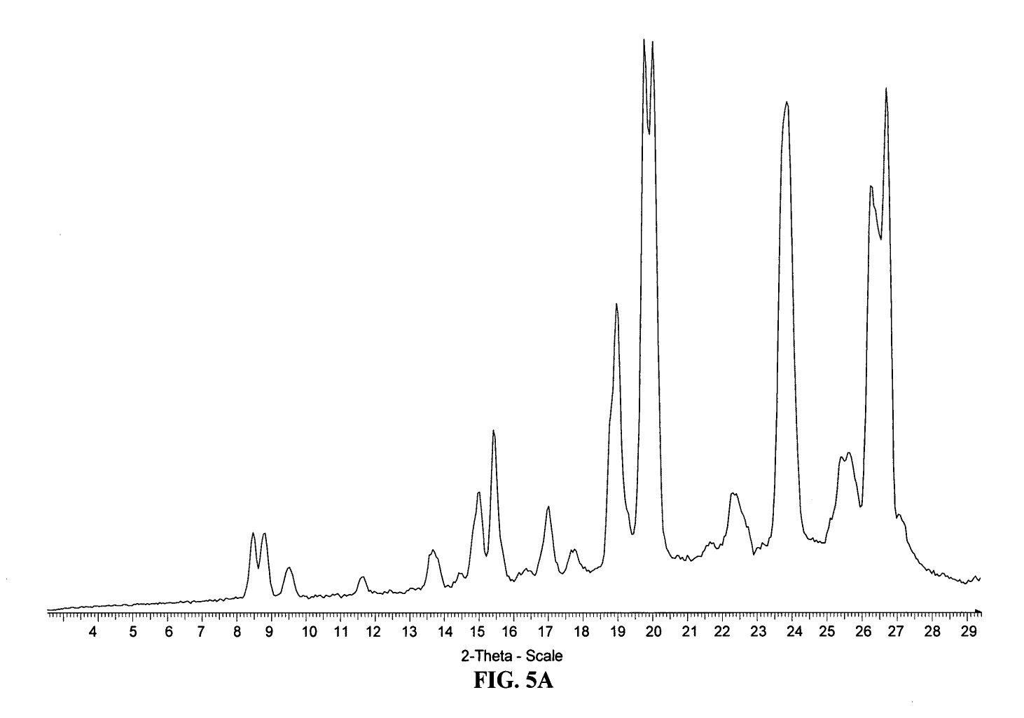

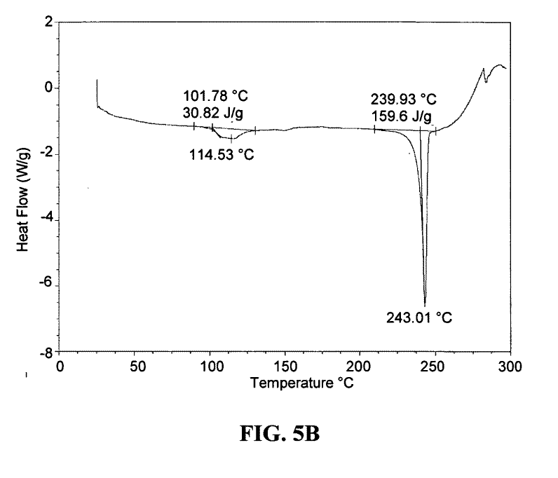

A sample of 1 in crystal form F was prepared by recrystallization from methyl ethyl ketone (MEK). The sample was characterized by XRPD and DSC. FIG. 5A shows an XRPD trace of crystal form F. The XRPD pattern of crystal form F is characterized by peaks at 2θ of 8.44°, 8.78°, 9.48°, 11.60°, 13.66°, 14.94°, 15.43°, 17.00°, 17.70°, 18.94°, 19.76°, 20.00°, 22.35°, 23.83°, 25.40°, 25.62°, 26.26°, and 26.68°. FIG. 5B shows a DSC thermogram for crystal form F. DSC thermograms of crystal form E show a desolvation endotherm above 102° C., and a minimum in (i.e., melting point) at about 240° C. The melting point of desolvated form F suggests that form F converts to form A upon desolvation.

### Example 10

### Characterization of Crystal Form G of 1 (hexafluoroisopropanol solvate)

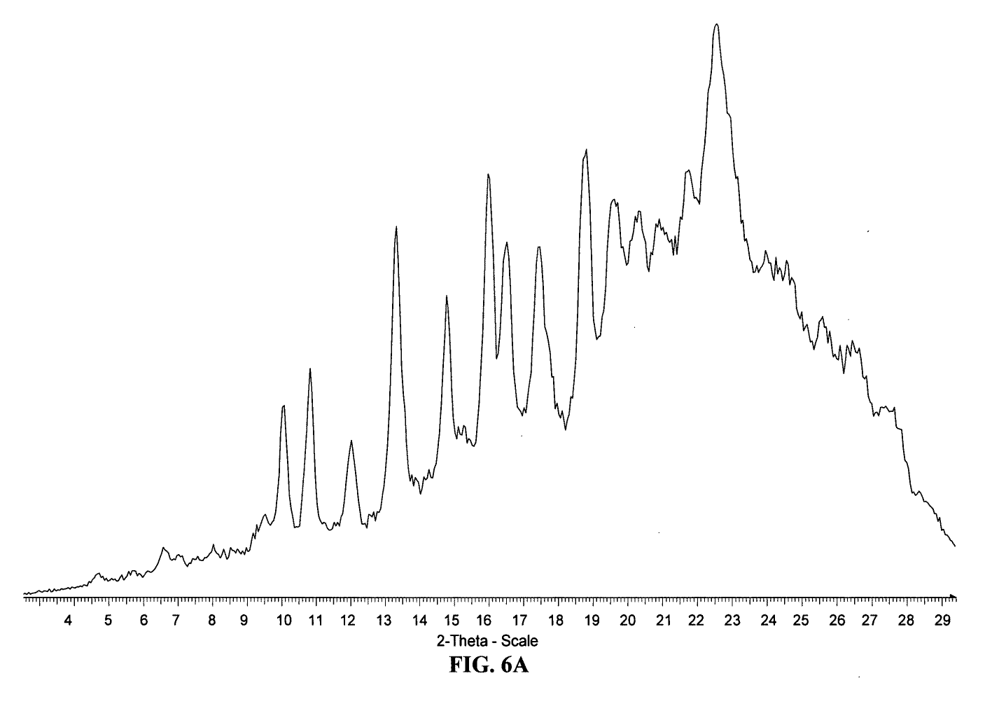

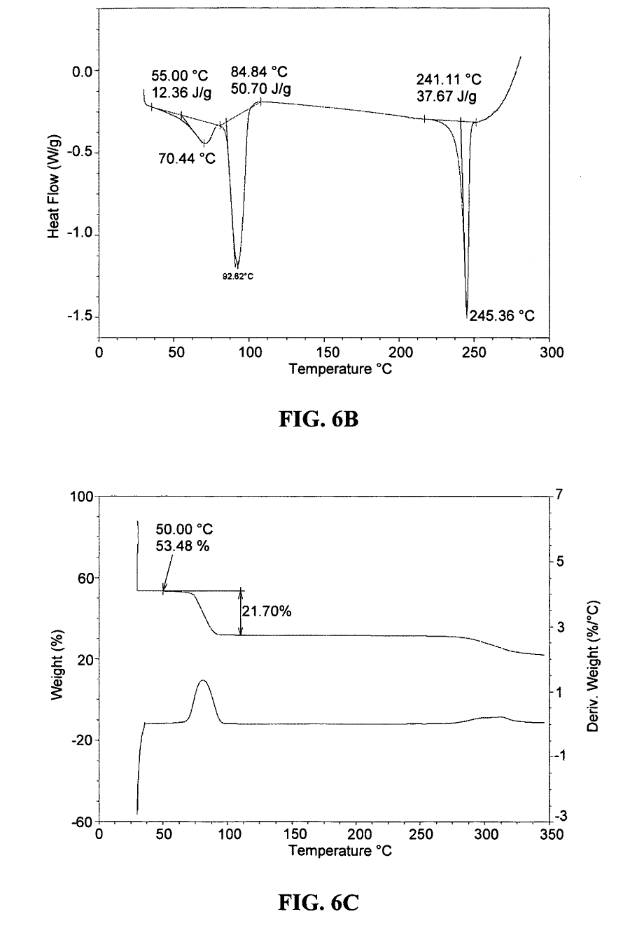

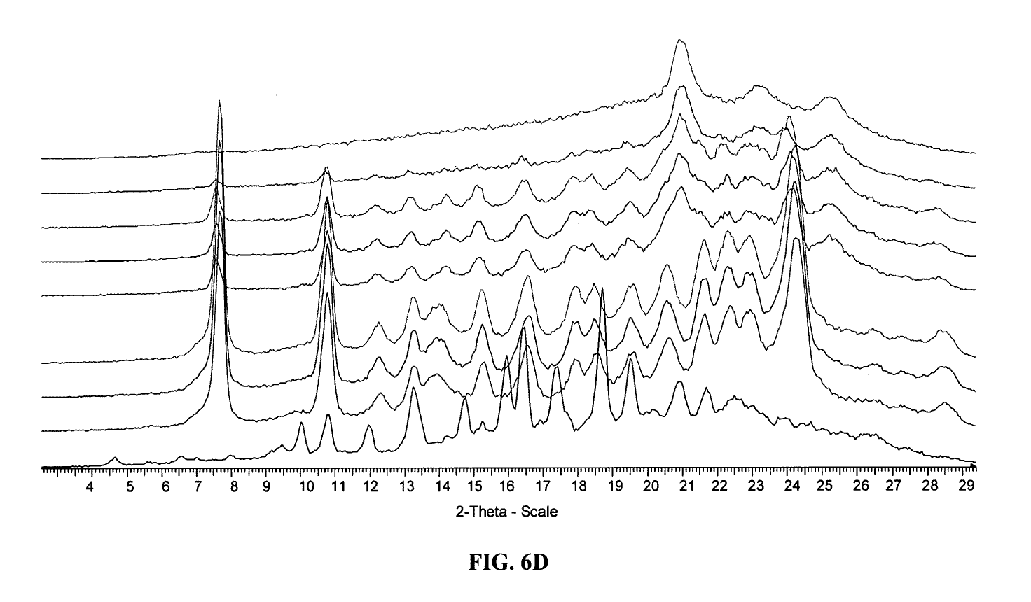

A sample of 1 in crystal form G was prepared by recrystallization from 1,1,1,3,3,3-hexafluoropropan-2-ol. The sample was characterized by XRPD, DSC, and TGA. FIG. 6A shows an XRPD trace of crystal form G. The XRPD pattern of crystal form G is characterized by peaks at 2θ of 4.66°, 6.56°, 10.06°, 10.81°, 12.00°, 13.31°, 14.74°, 16.00°, 16.51°, 17.40°, 18.79°, 19.56°, 20.31°, 21.71°, and 22.59°. FIG. 6B shows a DSC thermogram for crystal form G. DSC thermograms of crystal form G show a desolvation endotherm above 84° C., and a minimum in (i.e., melting point) at about 241° C. The melting point of desolvated form G suggests that form G converts to form A upon desolvation.

FIG. 6C shows a TGA trace for crystal form G. TGA revealed that form G lost 40% of its weight upon heating from ambient temperature to 50° C., or 1.3 moles solvent per mole of 1, consistent with form G being a hexafluoroisopropanol solvate.

FIG. 6D shows variable temperature XRPD traces of crystal form G. Traces (from bottom to top) were recorded at ambient temperature, 25° C., 70° C., 100° C., 120° C., 210° C., 220° C., 230° C., 235° C., and 240° C. Note that the traces recorded above 200° C. showed additional signals due to the presence of a protective semi-transparent dome. The final product was crystal form B.

### Example 11

### Recrystallization from Various Solvents

500 μL of each of 24 solvents were added to 50 mg±5 mg of form A, to produce a saturated solution. If complete dissolution was achieved, additional material was added until an excess of solid was present.

The vials were capped and placed in a shaking incubator which cycled between ambient temperature and 50° C., changing every 12 hours. Shaking was continued for 4 days. Inspection of the vials showed that the majority of the 1,1,1,3,3,3-hexafluoropropan-2-ol had evaporated, so an additional 500 μL was added. Inspection after another 2 days showed that this vial now contained only a solution, so additional solid (˜30 mg) was added.

A sample of each slurry was transferred to a glass slide, partially dried either by evaporation or by wicker filtration of any excess solvent and examined by XRPD. Recrystallization from 1,1,1,3,3,3-hexafluoro-propan-2-ol afforded crystal form G (see above). Under these conditions, recrystallization from acetone, acetonitrile, THF, DMA, DCM, cyclohexane, heptane, n-butanol, DMF, 1,4-dioxane, ethyl acetate, ethanol, butyl acetate, i-propyl acetate, MEK, methanol, MIBK, propan-1-ol, propan-2-ol, t-BME, toluene, water, or NMP afforded crystal form A.

### Example 12

### Characterization of Crystal Form H of 1 (THF Solvate)

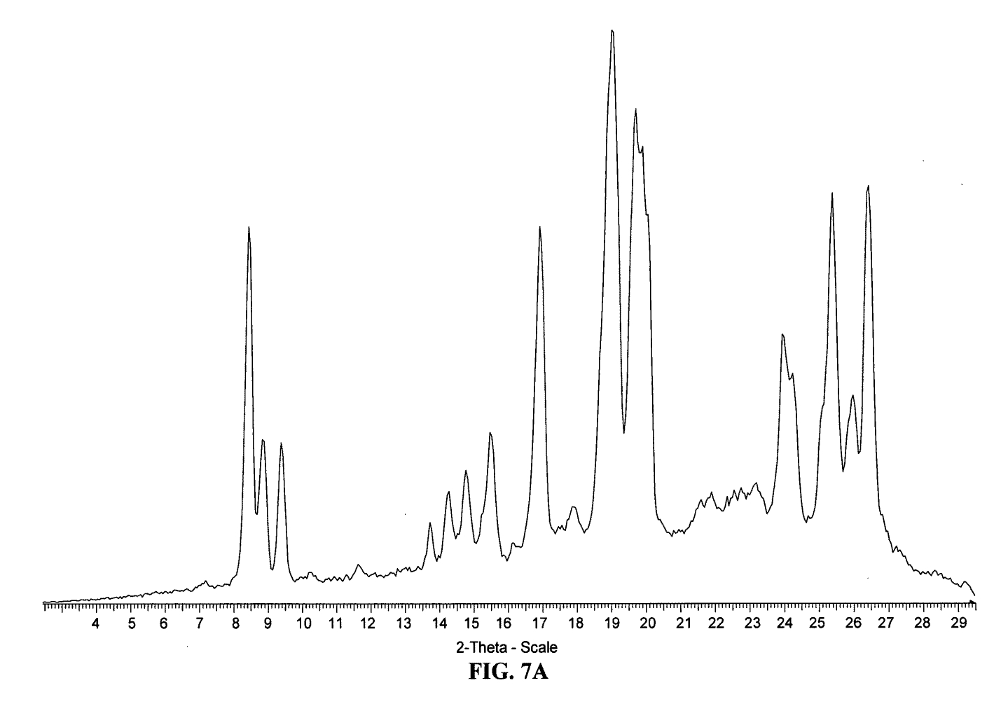

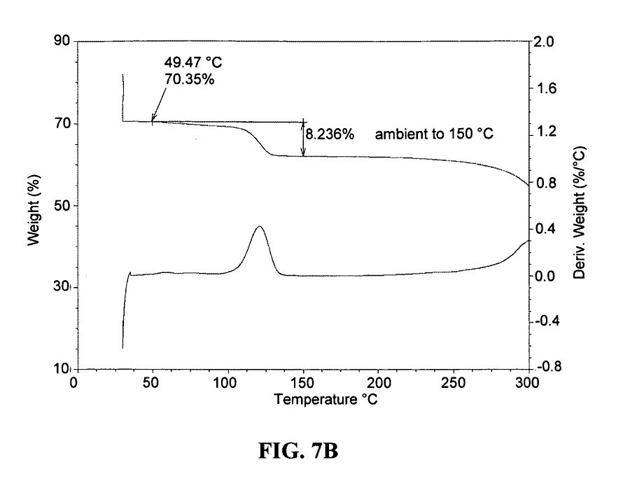

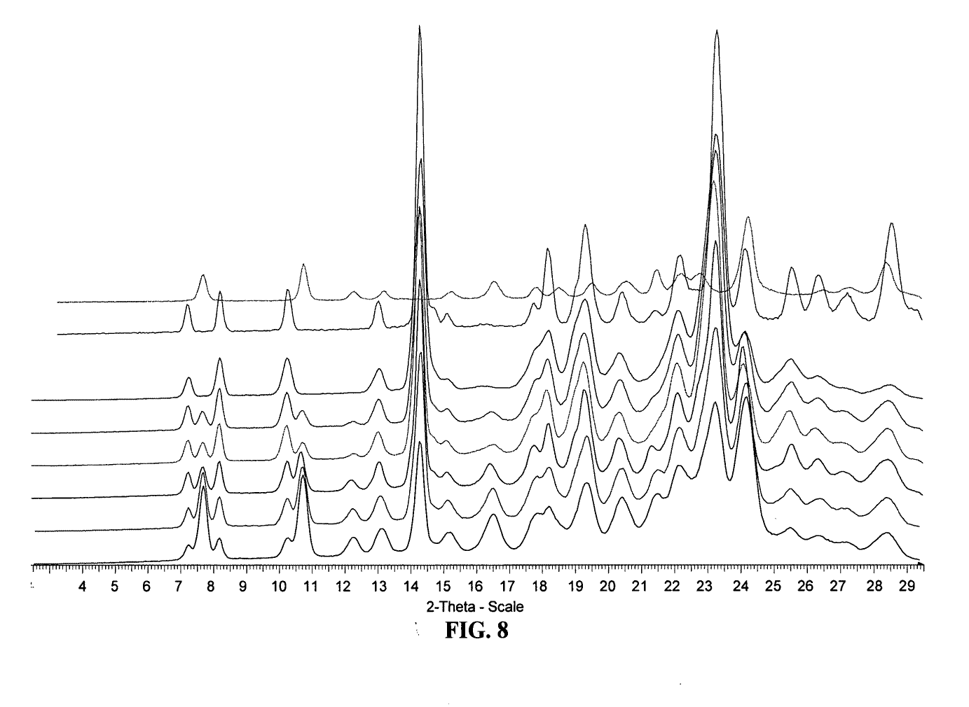

A sample of 1 in crystal form H was prepared by recystallization from THF. The sample was characterized by XRPD and TGA. FIG. 7A shows the XRPD trace of Form H, which is characterized by peaks at 2θ of 8.40°, 8.82°, 9.33°, 13.67°, 14.21°, 14.74°, 15.43°, 16.88°, 17.88°, 19.06°, 19.73°, 23.96°, 25.36°, 25.99°, and 26.45°. FIG. 7B shows a TGA trace for crystal form H. TGA revealed that form H lost 38% of its weight upon heating from ambient temperature to 150° C., consistent with form H being a THF hemisolvate.

### Example 13

### Relative Stability of Forms A and B

The relative stabilities of the forms A and B was determined by a vapour diffusion experiment. Approximately equal amounts of the two forms were ground together to produce an intimate mixture. The mixture was packed into a silicon 510-cut recessed wafer XRPD holder and the XRPD of the mixture determined. The holder was then placed in a covered dish containing NMP (a known solvent 1) at room temperature. The mixture was re-examined from time to time to monitor any changes in the XRPD pattern.

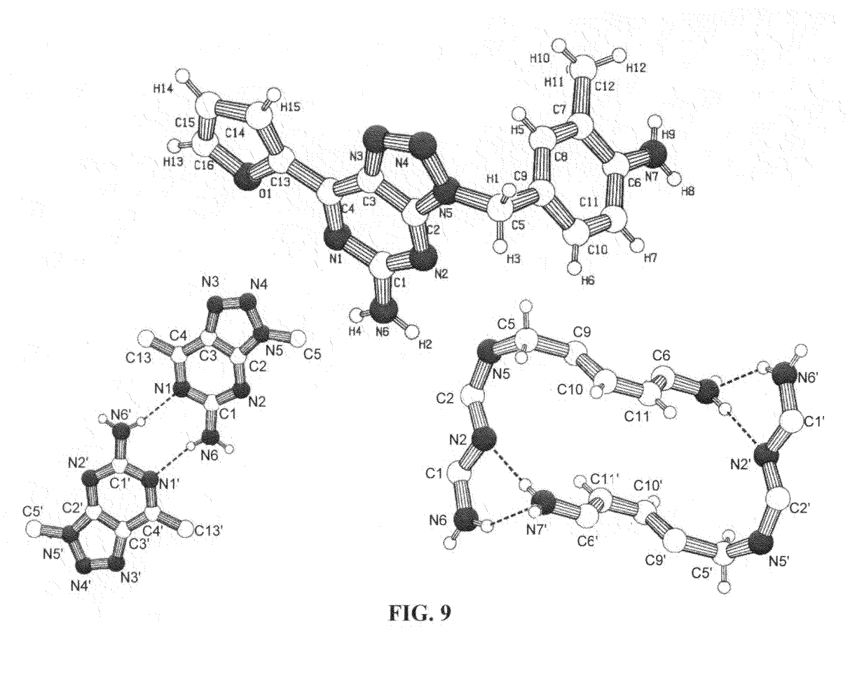

FIG. 8 shows that over time the peaks characteristic of Form B diminish, disappearing complete by the 23 day time point. This indicated that at room temperature, Form A was the more stable polymorph. The traces from bottom to top in FIG. 8 were recorded initially, at 24 hours, at 3 days, at 1 week, at 10 days, and at 23 days; the topmost traces are a form A reference and a form B reference.

### Example 14

### Relative Stability of Forms D and H

Forms D and H are both THF solvates and appear to have similar stoichiometry. Although they have very similar XRPD patterns (compare FIG. 3A with FIG. 7A), they were readily distinguished by TGA, having significantly different desolvation temperatures (compare FIG. 3B with FIG. 7B).

The relative stabilities of Forms D and H was investigated by a vapour diffusion experiment. Approximately equal amounts of the two forms were combined to produce an intimate mixture. The mixture was packed into a silicon XRPD holder and the XRPD of the mixture determined. The holder was then placed in a covered dish containing THF:NMP approx. 90:10 v/v at room temperature. The mixture was re-examined from time to time to monitor any changes in the XRPD pattern.

There was been no significant change in the XRPD traces over 12 days. Although the XRPD results were inconclusive, because Form H had the higher desolvation temperature, it was more likely to be the stable form of the THF solvate.

### Example 15

### Crystal Structure of Form A

The crystal structure of form A of 1 was solved using powder diffraction data. Form A produced monoclinic crystals in which the asymmetric unit is C₁₆H₁₅N₇O₁ (Z′=1), the space group is P2₁/a, and a=24.7948(6) Å, b=12.0468(2) Å, c=4.9927(1) Å, β=90.959(1)°, V=1491.1 Å³, T=293 K. The structure was solved using the global optimization methodology implemented in the program DASH, using diffraction data collected to a resolution of ≈2 Å. The structure obtained was consistent with the diffraction data and the presence of a small degree of preferred orientation in the sample was detected and allowed for. Table 1 presents the atomic coordinates of form A.

TABLE 1
Atomic coordinates of form A

| Atom | x | y | z |
|---|---|---|---|
| N1 | 0.06962 | 0.45383 | 0.16817 |
| C1 | 0.03914 | 0.38655 | 0.31579 |
| N2 | 0.05427 | 0.31306 | 0.49964 |
| C2 | 0.10785 | 0.31365 | 0.53380 |
| C3 | 0.14440 | 0.38040 | 0.40175 |
| C4 | 0.12320 | 0.45298 | 0.20222 |
| H1 | 0.15075 | 0.14910 | 1.00641 |
| N3 | 0.19528 | 0.35679 | 0.49370 |
| N4 | 0.19226 | 0.28023 | 0.67462 |
| N5 | 0.13872 | 0.25191 | 0.70216 |
| C5 | 0.12202 | 0.16549 | 0.88446 |
| H2 | −0.03843 | 0.35639 | 0.34313 |
| N6 | −0.01402 | 0.39760 | 0.25618 |
| H3 | 0.09164 | 0.19026 | 0.98130 |
| H4 | −0.02498 | 0.44618 | 0.12958 |
| C6 | 0.07911 | −0.12980 | 0.44550 |
| C7 | 0.12911 | −0.07727 | 0.39779 |
| C8 | 0.14244 | 0.01757 | 0.53925 |
| C9 | 0.10704 | 0.06046 | 0.72836 |
| C10 | 0.05771 | 0.01026 | 0.77547 |
| C11 | 0.04416 | −0.08433 | 0.63470 |
| N7 | 0.06402 | −0.22226 | 0.30766 |
| C12 | 0.16656 | −0.12598 | 0.19504 |
| H5 | 0.17509 | 0.05312 | 0.50899 |
| H6 | 0.03423 | 0.04002 | 0.89982 |
| H7 | 0.01125 | −0.11872 | 0.66579 |
| H8 | 0.03330 | −0.25250 | 0.33665 |
| H9 | 0.08521 | −0.25047 | 0.19142 |
| H10 | 0.20001 | −0.08581 | 0.19868 |
| H11 | 0.15033 | −0.12069 | 0.01947 |
| H12 | 0.17330 | −0.20260 | 0.23733 |
| C13 | 0.15676 | 0.52613 | 0.04193 |
| C14 | 0.21025 | 0.55261 | 0.02784 |
| C15 | 0.21529 | 0.63412 | −0.18506 |
| C16 | 0.16575 | 0.65038 | −0.28124 |
| O1 | 0.12885 | 0.58773 | −0.15264 |
| H13 | 0.15667 | 0.70268 | −0.43089 |
| H14 | 0.24918 | 0.67045 | −0.24638 |
| H15 | 0.24018 | 0.52181 | 0.14212 |

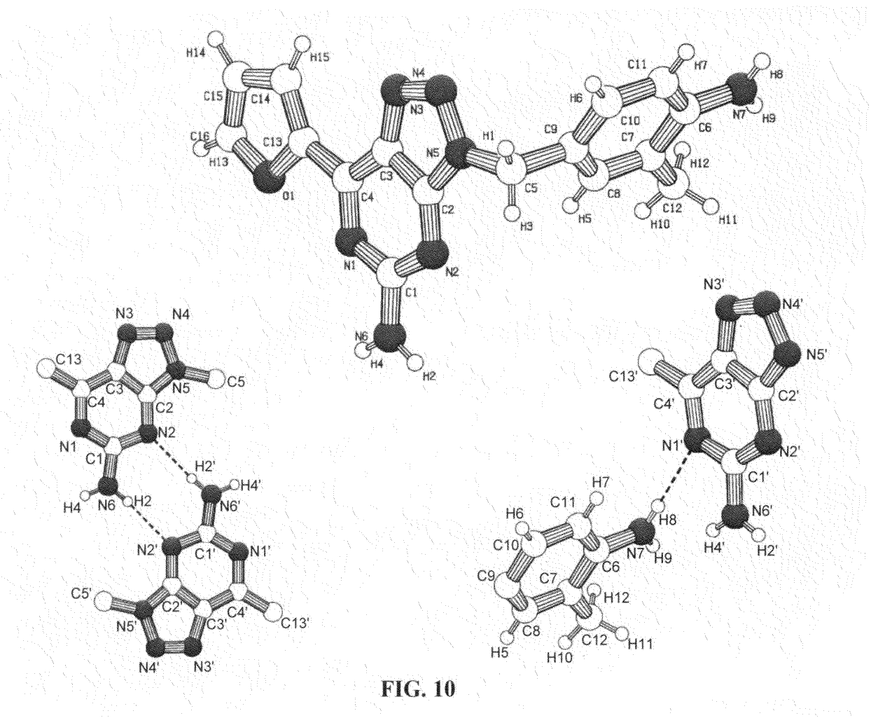

FIG. 9 shows three views of 1 in form A based on the crystal structure: at top, the full structure; at bottom left, hydrogen bonding involving N1 and N6; at bottom right, hydrogen bonding involving N7, N2 and N6.

### Example 16

### Crystal Structure of Form B

The crystal structure of form B of 1 was solved using powder diffraction data. Form B produced monoclinic crystals in which the asymmetric unit was C₁₆H₁₅N₇O₁ (Z′=1), the space group is P2₁/c with lattice constants a=11.6824 (6) Å, b=16.4814 (2) Å, c=8.0829 (1) Å, β=96.9979 (1)°, V=1544.7 Å³, T=293 K. The structure was solved using the global optimization methodology implemented in the program DASH, using diffraction data collected to a resolution of ≈2 Å. The structure obtained was consistent with the diffraction data. No preferred orientation was detected in the sample. Table 2 presents the atomic coordinates of form B.

TABLE 2
Atomic coordinates of form B

| Atom | x | y | z |
|---|---|---|---|
| N1 | −0.10556 | 0.19750 | 0.07525 |
| C1 | −0.05677 | 0.12819 | 0.03355 |
| N2 | 0.03730 | 0.11740 | −0.04030 |
| C2 | 0.08246 | 0.18869 | −0.07725 |
| C3 | 0.04012 | 0.26507 | −0.04623 |
| C4 | −0.05904 | 0.26844 | 0.03911 |
| H1 | 0.27417 | 0.16079 | −0.31749 |
| N3 | 0.10985 | 0.32245 | −0.10348 |
| N4 | 0.19191 | 0.28615 | −0.16837 |
| N5 | 0.17713 | 0.20355 | −0.15306 |
| C5 | 0.25753 | 0.14592 | −0.20942 |
| H2 | −0.08930 | 0.01214 | 0.05745 |
| N6 | −0.11431 | 0.06222 | 0.07918 |
| H3 | 0.22417 | 0.09327 | −0.21366 |
| H4 | −0.17760 | 0.06841 | 0.13125 |
| C6 | 0.57356 | 0.14375 | 0.12514 |
| C7 | 0.46976 | 0.11831 | 0.18308 |
| C8 | 0.36887 | 0.11906 | 0.07464 |
| C9 | 0.36951 | 0.14520 | −0.08989 |
| C10 | 0.47086 | 0.16967 | −0.14863 |
| C11 | 0.57181 | 0.16897 | −0.04104 |
| N7 | 0.67507 | 0.14274 | 0.22691 |
| C12 | 0.47162 | 0.09128 | 0.36114 |
| H5 | 0.30015 | 0.10218 | 0.11100 |
| H6 | 0.47055 | 0.18622 | −0.25868 |
| H7 | 0.64005 | 0.18556 | −0.07924 |
| H8 | 0.73756 | 0.15749 | 0.18947 |
| H9 | 0.67674 | 0.12731 | 0.32876 |
| H10 | 0.39418 | 0.08132 | 0.38436 |
| H11 | 0.51602 | 0.04231 | 0.37864 |
| H12 | 0.50582 | 0.13293 | 0.43433 |
| C13 | −0.11174 | 0.34435 | 0.08418 |
| C14 | −0.09261 | 0.42549 | 0.06565 |
| C15 | −0.18122 | 0.46808 | 0.14310 |
| C16 | −0.24562 | 0.41097 | 0.20115 |
| O1 | −0.20802 | 0.33536 | 0.16942 |
| H13 | −0.31384 | 0.42231 | 0.26120 |
| H14 | −0.19147 | 0.52814 | 0.15098 |
| H15 | −0.02969 | 0.45072 | 0.00956 |

FIG. 10 shows three views of 1 in form B based on the crystal structure: at top, the full structure; at bottom left, hydrogen bonding involving N2 and N6; at bottom right, hydrogen bonding involving N7 and N1.

A comparison of FIGS. 9 and 10 highlights differences in the molecular conformation observed in the two forms with respect to the orientation of ring (C6-C7-C8-C9-C10-C11). Compare, e.g., the position of methyl carbon C12 in FIGS. 9 and 10. In terms of packing, forms 1 and 2 have in common the dimer motif (i.e., hydrogen bonding involving a pyrimidine nitrogen and the 5-amino group (N6)) and a propensity for face-to-face close-packing of planar ring structures. However, a comparison of the same figures also reveals differences in intermolecular hydrogen bonding between the two forms (i.e., N6-N1′, N7-N6′, N7-N2′ in form A; N6-N2′, N7-N1′ in form B). Form A has the higher density (V=1491.1 Å³, compared with 1544.7 Å³ for form 2), consistent with the observation that form A is the more thermodynamically more stable of the two forms.

Other embodiments are within the scope of the following claims.

What is claimed is:

1. A composition comprising crystal form B of 3-(4-amino-3-methylbenzyl)-7-(furan-2-yl)-3H-[1,2,3]triazolo[4,5-d]pyrimidin-5-amine.

2. The composition of claim 1, wherein the composition is substantially pure crystal form B of 3-(4-amino-3-methylbenzyl)-7-(furan-2-yl)-3H-[1,2,3]triazolo[4,5-d]pyrimidin-5-amine.

3. The composition of claim 2, wherein the composition is characterized by peaks in X-ray powder diffraction at 2θ of 7.64°, 10.70°, 12.23°, 21.46°, 22.25°, 22.79°, 24.25°, and 28.43°.

4. The composition of claim 2, wherein the composition is characterized by peaks in X-ray powder diffraction at 2θ of 7.64°, 10.70°, 12.23°, 13.17°, 15.24°, 16.50°, 17.82°, 18.50°, 19.49°, 20.52°, 21.46°, 22.25°, 22.79°, 24.25°, 26.50°, 27.33°, and 28.43°.

5. The composition of claim 1, further comprising a pharmaceutically acceptable carrier.

6. A composition comprising a solvate of 3-(4-amino-3-methylbenzyl)-7-(furan-2-yl)-3H-[1,2,3]triazolo[4,5-d]pyrimidin-5-amine.

7. The composition of claim 6, wherein the composition comprises a THF solvate, a methyl ethyl ketone solvate, a 1,4-dioxane solvate, or a 1,1,1,3,3,3-hexafluoropropan-2-ol solvate of 3-(4-amino-3-methylbenzyl)-7-(furan-2-yl)-3H-[1,2,3]triazolo[4,5-d]pyrimidin-5-amine.

8. The composition of claim 7, wherein the solvate is substantially pure.

9. The composition of claim 8, wherein the solvate is crystal form D of 3-(4-amino-3-methylbenzyl)-7-(furan-2-yl)-3H-[1,2,3]triazolo[4,5-d]pyrimidin-5-amine.

10. The composition of claim 8, wherein the solvate is crystal form E of 3-(4-amino-3-methylbenzyl)-7-(furan-2-yl)-3H-[1,2,3]triazolo[4,5-d]pyrimidin-5-amine.

11. The composition of claim 8, wherein the solvate is crystal form F of 3-(4-amino-3-methylbenzyl)-7-(furan-2-yl)-3H-[1,2,3]triazolo[4,5-d]pyrimidin-5-amine.

12. The composition of claim 8, wherein the solvate is crystal form G of 3-(4-amino-3-methylbenzyl)-7-(furan-2-yl)-3H-[1,2,3]triazolo[4,5-d]pyrimidin-5-amine.

13. The composition of claim 8, wherein the solvate is crystal form H of 3-(4-amino-3-methylbenzyl)-7-(furan-2-yl)-3H-[1,2,3]triazolo[4,5-d]pyrimidin-5-amine.

14. A method of preparing crystal form B of 3-(4-amino-3-methylbenzyl)-7-(furan-2-yl)-3H-[1,2,3]triazolo[4,5-d]pyrimidin-5-amine comprising contacting 3-(4-amino-3-methylbenzyl)-7-(furan-2-yl)-3H-[1,2,31]triazolo[4,5-d]pyrimidin-5-amine, an N-protected derivative thereof, or a combination thereof, with a sulfonic acid.

15. The method of claim 14, wherein the sulfonic acid is methanesulfonic acid.

16. The method of claim 14, wherein contacting with a sulfonic acid includes contacting with an aqueous solution of methanesulfonic acid having a concentration of 1 M or greater.

17. The method of claim 14, wherein the N-protected derivative of 3-(4-amino-3-methylbenzyl)-7-(furan-2-yl)-3H-[1,2,3]triazolo[4,5-d]pyrimidin-5-amine is 3-(4-trifluoroacetamido-3-methylbenzyl)-7-(furan-2-yl)-3H-[41,2,3]triazolo[4,5-d]pyrimidin-5-amine.

18. The method of claim 14, further comprising contacting 3-(4-amino-3-methylbenzyl)-7-(furan-2-yl)-3H-[1,2,3]triazolo[4,5-d]pyrimidin-5-amine, an N-protect derivative thereof, or a combination thereof, with a basic composition.

19. The method of claim 18, wherein the basic composition is an aqueous potassium hydroxide solution.

20. The method of claim 19, wherein the concentration of potassium hydroxide in the aqueous potassium hydroxide solution is greater than 1 M.

21. A method of preparing crystal form B of 3-(4-amino-3-methylbenzyl)-7-(furan-2-yl)-3H-[1,2,3]triazolo[4,5-d]pyrimidin-5-amine comprising contacting 3-(4-amino-3-methylbenzyl)-7-(furan-2-yl)-3H-[1,2,3]triazolo[4,5-d]pyrimidin-5-amine with a carboxylic acid.

22. The method of claim 21, wherein the carboxylic acid is formic acid, acetic acid, trichloroacetic acid, trifluoroacetic acid, propionic acid, butanoic acid, or a combination thereof.

23. The method of claim 21, further comprising contacting 3-(4-amino-3-methylbenzyl)-7-(furan-2-yl)-3H-[1,2,3]triazolo[4,5-d]pyrimidin-5-amine with a basic composition.

24. The method of claim 23, wherein the basic composition is an aqueous ammonium hydroxide solution.
